SEQUENCE LISTING (4 sequences)

SEQ ID NO: 1 - DNA, 5314 bp, UNKNOWN
  bac SXT8
  attB1 recombination site (1)..(25)
  EV-A71 VP4-VP2-VP3(partial) (107)..(1660)
  CV-A16 VP3(partial) (1661)..(1801)
  CV-A16 VP1 (1802)..(2695)
  IRES (2696)..(3281)
  EV-A71 3C-3D (3282)..(5222)
  attB2 recombination site (5289)..(5314)
acaagtttgtacaaaaaagcaggctagatcttaatacgactcactatattaattaaggatcccaaaaaattgaaattttattttttttttttggaatataaataatatgggctcgcaagtcagcactcagaggtcgggctcacacgaaaacagcaactcggcaacggaaggctcaactatcaactacaccactatcaactactacaaggacagttacgctgccaccgcaggcaagcagtcgctgaaacaagaccctgataagttcgctaaccccgtgaaagacatcttcactgaaatggcagcgccactcaagagtccgtcggcggaggcttgcggatactctgatcgtgtcgctcagttgaccattggtaactcaactatcaccactcaagaagctgccaacatcattgtcggctacggagagtggccctcttactgttccgacagcgatgccaccgcagttgacaagcctacccgccctgatgtgtcagtcaacagattctacacactggacacgaagctctgggaaaagtccagcaaaggctggtactggaaattcccagatgtgttgacagagacgggagtcttcggtcagaacgcccaattccactacctgtacaggagtggattctgcatccacgtgcagtgtaacgcttcgaagttccatcaaggtgccctgctcgttgcagtgctgcctgaatacgtcatcggaaccgttgccggtggcaccggtactgaggacacacaccctccctacaaacagactcaacctggtgccgacggattcgagctgcagcatccatacgtcctcgatgctggaatccctatttcccagctgaccgtttgcccccaccaatggattaacctccgcacaaacaactgtgctacgatcattgtgccatacatcaacgcattgccgttcgactccgcgctgaaccattgcaacttcggtttgctggtggtccctatcagccccctggactacgatcagggcgcaaccccagtgatcccgattacaatcacgttggcgcctatgtgttctgagttcgctggactgaggcaggcagtcacccaaggattccctactgagttgaagcccggtactaaccaattcctgacaacggacgatggagtcagcgctcctatcctgcccaacttccaccctacaccctgcattcatatccccggtgaagttcgtaacctcttggagctgtgtcaggtggagaccatcctcgaagttaacaacgtgccaaccaacgctacttccttgatggaaaggctgagattccctgtgagcgctcaagctggcaagggtgagctgtgcgctgtcttcagggctgacccaggtcgtaacggaccgtggcagagcacactgctgggacaactctgtggatactacacgcagtggtctggttcattggaagtgaccttcatgttcactggctccttcatggcaacaggaaagatgttgatcgcgtacacgccaccgggaggtccattgccgaaagaccgcgccacagcaatgctgggcacgcacgtcatctgggatttcggactccaatcttcagtcaccttggttattccttggatctccaacactcactacagggcgcatgctagagacggtgtcttcgattactacaccactggcctggtgtccatatggtaccagacgaactacgtcgttcctatcggagctcccacaacggcctacattgtggcactggcagcggctcaagacaacttcaccatgaagctctgcaaagacacagaggatatcgaacagacggcgaacattcaaggtgaccccatcgctgacatgattgatcagacagtcaacaaccaagttaaccgctccctgactgctctccaggtgttgcctactgcagcaaacactgaggcctcttcacaccgtctcggcacaggagtggtccccgcattgcaagcggctgaaaccggagcgagttcgaacgcttccgacaagaacctgatcgagacccgctgcgtcctcaaccaccatagcacacaggaaacggccattggaaacttcttctctcgtgcaggtctggtttcaatcattactatgcctaccactggcacccagaacactgacggatacgtcaactgggacatcgatctcatgggctacgctcaattgcgccgtaagtgtgagctgttcacctacatgcgcttcgatgccgaattcactttcgttgtggcaaaacctaacggagagctggtgccccagctgctccaatacatgtacgtccctcccggtgctcctaagccaacttcccgtgacagcttcgcctggcagaccgcaactaaccctagtgttttcgtgaaaatgacagatccaccggcccaggtctcggttccattcatgtctccggcatcagcgtaccaatggttctacgacggttaccccaccttcggcgagcacctccaggctaacgacttggattacggccaatgcccaaacaacatgatgggaaccttctctatcaggacagtgggtacggaaaagagtccccattcgattactctgagggtctacatgagaatcaaacacgttagagcctggattccaaggccgctcagaaaccagccatacttgttcaagactaaccctaactacaaaggcaacgatattaagtgtacctcgactagccgtgataaaattactactctgtaagcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaatgggtccaagtcttgactttgctctttccctgctgagaaggaacatcaggcaagtccaaacagatcagggccattttaccatgttgggtgtcagggatcgcttggctgttctcccaaggcacgcacagcccggcaagactatttgggtggagcacaaacttgtgaacgtcctcgacgcaatcgagctggtggatgaacagggcgttaatttggaactcacactggtgacacttgacactaatgaaaaatttagagatatcaccaagttcattccagagaccattagtggcgctagtgatgcaactttggtgatcaatacagaacatatgccatcaatgtttgtcccagtgggcgacgtcgtgcagtatggattcttgaaccttagtggaaagccaacacataggaccatgatgtacaatttccctacaaaagcaggacaatgtggaggtgtggtcacatccgtcggtaagatcgttggtattcacattggcggcaacggccgccaaggtttctgtgctggtttgaagaggagttacttcgcaagtgtgcagggtgagatccaatgggtgaaacctaacaaagaaactggtagactgaacatcaatggaccaactcgcactaagctggagcctagtgtgtttcatgaagtgtttgaaggcaataaggaaccagcagtcttgacaagtaaagaccctagactggaggtcgactttgagcaagccctgttttccaagtatgtgggcaatgttttgcatgagcccgatgaatacgtgactcaagctgccctccactatgcgaatcaactcaaacagttggacataaacactagcaagatgagcatggaggaagcgtgctatggcactgagaacctggaagcaattgatctctgtactagtgccggatatccatacagcgcccttggcatcaagaaaagagacattctcgaccccgtaaccagggatgtgtctaagatgaaattctatatggacaaatacggcctcgatctgccatactccacctacgtaaaggatgagcttcgctctctggataaaatcaagaaaggaaagtcacgcctgatagaggctagtagcttgaatgactctgtctacctcagaatgacttttggccacctttacgaggtatttcatgctaaccctggcactgtgaccggttccgcagtgggttgcaacccagacgtgttttggagtaaacttccgatccttctgcctggctcactctttgcctttgactactcaggatatgatgctagcctcagcccggtatggttcagggcccttgaaattgtgttgcgcgaaattggctatccagaagaggccgtgtcccttatagaaggaatcaaccacactcaccacgtgtaccgcaacaaaacatactgtgtacttggtggcatgccctcaggctgttctggtacttccatctttaactcaatgatcaacaacatcatcattagaaccctcttgatcaaaacctttaagggaatagacctggatgagttgaacatggtggcctatggcgacgatgtgttggctagttatccctttcctatagattgccttgagttggctaagactggcaaagagtatggtttgactatgacgcctgcagacaaatcaccctgtttcaatgaagtgacgtgggaaaatgctaccttccttaagagaggcttcttgccagaccaccaatttccattcttgattcaccctacgatgcccatgagagagatccatgagtccattcgctggactaaagacgcacgcaatacccaagaccacgtgcgctccctgtgtctgttggcatggcacaacggtaaggatgaatatgagaaatttgtgagtgcaattagatcagttccagttggaaaagcgttggccattcctaattttgagaatttgagaagaaattggctcgaattgttttaaatcgatttaattaatagcataaccccttggggcctctaaacgggtcttgaggggttttttggaattcacccagctttcttgtacaaagtggt

SEQ ID NO: 2 - DNA, 5314 bp, UNKNOWN
  bacSXT7
  attB1 recombination site (1)..(25)
  CV-A16 VP4-VP2-VP3(partial) (107)..(1660)
  EV-A71 VP3(partial) (1661)..(1801)
  EV-A71 VP1 (1802)..(2695)
  IRES (2696)..(3281)
  EV-A71 3C-3D (3282)..(5222)
  attB2 recombination site (5289)..(5314)
acaagtttgtacaaaaaagcaggctagatcttaatacgactcactatattaattaaggatcccaaaaaattgaaattttattttttttttttggaatataaataatatgggctcacaagtcagcacgcaacgcagcggaagccacgagaacagcaactcggcatcggaaggctctactatcaactacactacaatcaactactacaaggacgcgtacgctgccagtgctggtaggcaggacatgtcgcaagatcctaagaaattcaccgaccccgtgatggatgtcatccatgagatggctcctcccttgaagtccccaagcgccgaagcatgcggctactccgacagagtggcccagctgacaatcggaaacagcacgattaccactcaagaggcagcgaacatcgtgattgcttacggcgagtggccagaatactgtccggacacagatgcgacggctgtcgacaagcctacccgccctgatgtctccgttaacagattcttcaccctggacactaagtcctgggccaaggatagcaaaggatggtactggaaattccctgacgtgctcactgaagtgggtgtcttcggccagaacgctcaattccactacttgtaccgttctggcttctgcgttcatgtgcagtgtaacgcctcaaagttccaccaaggagcactgctcgtcgcggttctgccagagtacgtcctcggtaccatcgctggtggcactggcaacgaaaacagccatccaccgtacgcgacaacgcagcctggacaagttggtgctgtgctgactcacccctacgtcctcgacgccggtatcccattgtctcagctgacagtttgccctcatcaatggattaacctgaggacaaacaactgtgctacgatcattgtgccatacatgaacaccgtcccgttcgactctgccctgaaccactgcaacttcggattgctggtcatcccagtggtcccgctcgatttcaacgccggtgcaacttcagagatcccaattacagtgacgatcgccccgatgtgtgctgaattcgctggactgaggcaggcagtcaagcaaggaatccctaccgagttgaaacccggtactaaccagttcctgaccactgacgatggcgtgagcgctcctatcctccccggattccaccccacccctcccatccatattccaggagaggtccacaacctcttggaaatctgcagggttgagacaattttggaagtgaacaacctgaagacgaacgagacaacgcctatgcagagattgtgcttccccgtgagtgtccaatcgaaaacaggcgaactgtgcgctgccttccgcgctgacccaggccgtgatggaccttggcagagtaccatcctcggtcaattgtgtcgctactacacccagtggtctggctcactcgaagtgactttcatgttcgccggatccttcatggcaaccggcaagatgttgatcgcctacactccaccgggaggtaacgtgcctgctgaccgtatcaccgctatgctgggtactcacgttatttgggatttcggcttgcaatccagcgtgacactggttgtgccatggatctcaaacactcactacagggctcatgctagggcaggatacttcgactactacaccactggtatcattaccatatggtatcagactaactacgttgtgcctatcggtgctcctaacacagcatacatcattgcgctcgcagcggctcaaaagaacttcaccatgaagctctgcaaagacgcttcagatattttgcagaccggtactatccaaggcgaccgcgtggctgatgtcattgagagttcgatcggtgacagtgtgtcgcgcgccctcactcatgcgttgcctgctcccacaggccagaacacgcaagtgtccagccaccgtctggatacaggcaaggtccctgctctgcaggctgcagaaattggagcttcttcaaacgccagtgacgagtcgatgatcgaaacacgctgcgtgctgaactcccatagcacggctgagacaacgctcgactccttcttcagccgtgccggattggtcggtgaaatcgatctccctttgaagggcaccactaaccccaacggctacgctaactgggacattgacatcaccggctacgcccagatgcgccgtaaggttgaactcttcacctacatgaggttcgacgctgagttcactttcgtggcctgtacacccacgggcgaagtcgttccacagttgctgcaatacatgttcgtgcctcctggtgctcctaagcctgactcccgcgagtccctggcatggcagaccgcgactaacccctctgtcttcgttaaactctcagatccaccggcccaggtgtctgtcccattcatgagtccggcctcggcataccaatggttctacgacggataccccaccttcggagagcacaagcaagaaaaggacctggagtacggagcttgcccaaacaacatgatgggtactttcagcgttaggacagtgggcacgtctaagtcaaaatacccattggttgttaggatctacatgagaatgaagcacgtgagagcttggattcctcgccccatgcgtaaccagaactacctgttcaaagctaaccctaactacgcaggcaacagtatcaagcccacgggagcatcaagaacggcaatcaccactttgtaagcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaatgggtccaagtcttgactttgctctttccctgctgagaaggaacatcaggcaagtccaaacagatcagggccattttaccatgttgggtgtcagggatcgcttggctgttctcccaaggcacgcacagcccggcaagactatttgggtggagcacaaacttgtgaacgtcctcgacgcaatcgagctggtggatgaacagggcgttaatttggaactcacactggtgacacttgacactaatgaaaaatttagagatatcaccaagttcattccagagaccattagtggcgctagtgatgcaactttggtgatcaatacagaacatatgccatcaatgtttgtcccagtgggcgacgtcgtgcagtatggattcttgaaccttagtggaaagccaacacataggaccatgatgtacaatttccctacaaaagcaggacaatgtggaggtgtggtcacatccgtcggtaagatcgttggtattcacattggcggcaacggccgccaaggtttctgtgctggtttgaagaggagttacttcgcaagtgtgcagggtgagatccaatgggtgaaacctaacaaagaaactggtagactgaacatcaatggaccaactcgcactaagctggagcctagtgtgtttcatgaagtgtttgaaggcaataaggaaccagcagtcttgacaagtaaagaccctagactggaggtcgactttgagcaagccctgttttccaagtatgtgggcaatgttttgcatgagcccgatgaatacgtgactcaagctgccctccactatgcgaatcaactcaaacagttggacataaacactagcaagatgagcatggaggaagcgtgctatggcactgagaacctggaagcaattgatctctgtactagtgccggatatccatacagcgcccttggcatcaagaaaagagacattctcgaccccgtaaccagggatgtgtctaagatgaaattctatatggacaaatacggcctcgatctgccatactccacctacgtaaaggatgagcttcgctctctggataaaatcaagaaaggaaagtcacgcctgatagaggctagtagcttgaatgactctgtctacctcagaatgacttttggccacctttacgaggtatttcatgctaaccctggcactgtgaccggttccgcagtgggttgcaacccagacgtgttttggagtaaacttccgatccttctgcctggctcactctttgcctttgactactcaggatatgatgctagcctcagcccggtatggttcagggcccttgaaattgtgttgcgcgaaattggctatccagaagaggccgtgtcccttatagaaggaatcaaccacactcaccacgtgtaccgcaacaaaacatactgtgtacttggtggcatgccctcaggctgttctggtacttccatctttaactcaatgatcaacaacatcatcattagaaccctcttgatcaaaacctttaagggaatagacctggatgagttgaacatggtggcctatggcgacgatgtgttggctagttatccctttcctatagattgccttgagttggctaagactggcaaagagtatggtttgactatgacgcctgcagacaaatcaccctgtttcaatgaagtgacgtgggaaaatgctaccttccttaagagaggcttcttgccagaccaccaatttccattcttgattcaccctacgatgcccatgagagagatccatgagtccattcgctggactaaagacgcacgcaatacccaagaccacgtgcgctccctgtgtctgttggcatggcacaacggtaaggatgaatatgagaaatttgtgagtgcaattagatcagttccagttggaaaagcgttggccattcctaattttgagaatttgagaagaaattggctcgaattgttttaaatcgatttaattaatagcataaccccttggggcctctaaacgggtcttgaggggttttttggaattcacccagctttcttgtacaaagtggt

SEQ ID NO: 3 - DNA, 5371 bp, UNKNOWN
  bacSXT11
  attB1recombination site (1)..(25)
  PV1 P1 (107)..(2749)
  IRES (2753)..(3338)
  EV-A71 3C-3D (3339)..(5279)
  attB2 recombination site (5346)..(5371)
acaagtttgtacaaaaaagcaggctagatcttaatacgactcactatattaattaaggatcccaaaaaattgaaattttattttttttttttggaatataaataatatgggagcacaggtctcatcacagaaggtcggagcacacgagaacagtaacagggcctacggaggaagtacaatcaactacaccaccatcaactactatcgcgactctgcctcaaatgccgctagcaagcaggactttagtcaggatccaagcaaattcactgagcccatcaaggacgtgctcattaaaacagcaccaatgctgaactcccccaatatcgaagcctgcggctactctgatagggtcctgcagctcaccctgggaaactcaactattaccactcaggaggcagccaatagtgtggtcgcatacggtcgctggcctgagtatctgagggactccgaagccaacccagtggaccagcccactgaacctgatgtcgctgcatgtagattctacacactggagaccgtgtcatggacaaaagaaagtaaaggctggtggtggaagctcccagacgctctgcgcgatatgggtctcttcggccagaacatgtactatcactacctgggaaggtcaggttacaccgtgcacgtccagtgcaatgccagtaaatttcatcagggcgcactcggagtgttcgccgtccctgagatgtgcctggccggagactctaacacaacccccactcatacatcataccagaacgcaaatcctggcgaaaagggcggaacctttactggaacattcaccccagacaacaatcagacaagccctgctcgcaggttttgcccagtggattacctgttcggaaacggtaccctgctcggaaatgctttcgtctttcctcaccagatcattaacctgaggacaaacaattgtgccaccctcgtgctgccatacgtcaatagtctgagcatcgactccatggtgaagcataacaattggggaattgctatcattccactggcacccctcaacttcgccaatgagagcagccctgaaatcccaattactctgacaatcgccccaatgtgctgtgagttcaacggtctgagaaatattaccctgccccgtctccagggtctgcctgtgatgaacacaccaggcagcaatcagtacctcaccgccgataactttcagtccccatgcgctctgcccgagttcgacgtgacccctcccatcgatattcccggcgaagtcaagaacatgatggagctggccgaaatcgacactatgattcctttcgatctgagtgctactaagaaaaatacaatggagatgtaccgggtgcagctgtcagacaaaccccacaccgacgatcctatcctctgcctgtccctctctcctgctaccgatccaaggctgagccacactatgctcggtgaaattctgaactactatacacattgggccggcagcctcaagttcacctttctgttctgtggatccatgatggctaccggtaaactgctcgtgtcttacgccccacccggtgctgaccctccaaagaaaagaaaggaggccatgctcggaacccacgtcatctgggatattggtctgcagtcttcatgcactatggtggtcccctggatctccaacactacataccgtcagactattgacgatagttttacagagggtggctacatcagcgtgttctatcagactcggattgtggtccccctctctacacctcgcgaaatggacatcctgggattcgtgagcgcctgtaacgacttctccgtcaggctgctcagagataccactcacatcgagcagaaggcaattgcccagggcctcggacagatgctggagagctccatcgacaacaccgtgagggaaactgtcggcgcttcaaccagtagagatgcactgccaaatactgaatcctctggacccgcacattctaaggagatccctgcactgaccgccgtggaaactggcgccacaaaccccctggtgcctagcgacaccgtccagactagacacgtgatccagcatcggagccgctccgagtcaagtgtggaatctttctttgctcgtggagcatgcgtcacaatcatgaccgtggacaactctgcttcaacaacctctaaggataaactgttctcagtgtggaagatcacatacaaagacaccgtccagctcaggcgtaagctggagttctttacctattcccgctttgatatggaatttactttcgtgatcacagctaacttcaccgagactaacaatggtcacgcactgaatcaggtctaccagattatgtatgtgccccctggcgcccctgtcccagaaaagtgggacgattacacttggcagacaagctccaacccctctatcttctacacctatggcactgctcccgcaagaatctcagtgccttatgtcggaatttccaatgcttactctcacttttatgacggtttcagcaaggtgcctctgaaagaccagagtgccgagctgggtgattccctgtacggcgccgcttccctcaacgacttcggaatcctggccgtgcgtgtggtcaacgatcacaatccaacaaaggtgacctccaaaatccgtgtctatctgaagccaaaacatattcgggtgtggtgtccacgtccaccccgggcagtcgcttactatggtcccggcgtggactataaggatggtactctcgcacctctgagcactaaggatctgactacctattaagcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaatgggtccaagtcttgactttgctctttccctgctgagaaggaacatcaggcaagtccaaacagatcagggccattttaccatgttgggtgtcagggatcgcttggctgttctcccaaggcacgcacagcccggcaagactatttgggtggagcacaaacttgtgaacgtcctcgacgcaatcgagctggtggatgaacagggcgttaatttggaactcacactggtgacacttgacactaatgaaaaatttagagatatcaccaagttcattccagagaccattagtggcgctagtgatgcaactttggtgatcaatacagaacatatgccatcaatgtttgtcccagtgggcgacgtcgtgcagtatggattcttgaaccttagtggaaagccaacacataggaccatgatgtacaatttccctacaaaagcaggacaatgtggaggtgtggtcacatccgtcggtaagatcgttggtattcacattggcggcaacggccgccaaggtttctgtgctggtttgaagaggagttacttcgcaagtgtgcagggtgagatccaatgggtgaaacctaacaaagaaactggtagactgaacatcaatggaccaactcgcactaagctggagcctagtgtgtttcatgaagtgtttgaaggcaataaggaaccagcagtcttgacaagtaaagaccctagactggaggtcgactttgagcaagccctgttttccaagtatgtgggcaatgttttgcatgagcccgatgaatacgtgactcaagctgccctccactatgcgaatcaactcaaacagttggacataaacactagcaagatgagcatggaggaagcgtgctatggcactgagaacctggaagcaattgatctctgtactagtgccggatatccatacagcgcccttggcatcaagaaaagagacattctcgaccccgtaaccagggatgtgtctaagatgaaattctatatggacaaatacggcctcgatctgccatactccacctacgtaaaggatgagcttcgctctctggataaaatcaagaaaggaaagtcacgcctgatagaggctagtagcttgaatgactctgtctacctcagaatgacttttggccacctttacgaggtatttcatgctaaccctggcactgtgaccggttccgcagtgggttgcaacccagacgtgttttggagtaaacttccgatccttctgcctggctcactctttgcctttgactactcaggatatgatgctagcctcagcccggtatggttcagggcccttgaaattgtgttgcgcgaaattggctatccagaagaggccgtgtcccttatagaaggaatcaaccacactcaccacgtgtaccgcaacaaaacatactgtgtacttggtggcatgccctcaggctgttctggtacttccatctttaactcaatgatcaacaacatcatcattagaaccctcttgatcaaaacctttaagggaatagacctggatgagttgaacatggtggcctatggcgacgatgtgttggctagttatccctttcctatagattgccttgagttggctaagactggcaaagagtatggtttgactatgacgcctgcagacaaatcaccctgtttcaatgaagtgacgtgggaaaatgctaccttccttaagagaggcttcttgccagaccaccaatttccattcttgattcaccctacgatgcccatgagagagatccatgagtccattcgctggactaaagacgcacgcaatacccaagaccacgtgcgctccctgtgtctgttggcatggcacaacggtaaggatgaatatgagaaatttgtgagtgcaattagatcagttccagttggaaaagcgttggccattcctaattttgagaatttgagaagaaattggctcgaattgttttaaatcgatttaattaatagcataaccccttggggcctctaaacgggtcttgaggggttttttggaattcacccagctttcttgtacaaagtggt

SEQ ID NO: 4 - DNA, 5335 bp, UNKNOWN
  bacSXT18
  attB1 recombination site (1)..(25)
  EV-A71 VP4-VP2-VP3-VP1(partial) (107)..(1832)
  PV1 VP1(partial) (1833)..(2713): A1832T and T1833C introduced a SacI restriction site. Double mutation leads to a Met to Ser amino acid substitution in PV1 VP1.
  IRES (2717)..(3302)
  EV-A71 3C-3D (3303)..(5243)
  attB2 recombination site (5310)..(5335)
acaagtttgtacaaaaaagcaggctagatcttaatacgactcactatattaattaaggatcccaaaaaattgaaattttattttttttttttggaatataaataatatgggctcacaggtgtccactcaacgctccggctcccatgaaaactctaattcagctacagaaggctccaccattaattacaccactattaactattacaaagattcctatgctgcgacagccggcaaacagagccttaaacaagaccctgataagtttgctaaccctgtcaaggacattttcactgaaatggctgcgcctcttaagtctccatccgctgaagcttgtggttacagtgatcgcgtggcacaactcaccattggaaactccaccattactacacaggaagcagcaaacatcatagtcggttatggtgaatggccctcatactgctccgatgacgacgctacagcggtggataagccaacgcgcccagatgtttccgtgaataggttttatacgttggacactaaactgtgggaaaagtcatccaagggctggtattggaagtttcctgatgtattgactgagaccggagtctttggccaaaacgcacaatttcactatttgtatagatcaggcttttgcatccatgtgcaatgtaacgctagcaagttccaccaaggagcgctgttggtcgctatacttccagagtatgtcataggcacggtggcaggcggtacaggaacagaggacagtcaccctccttacaaacaaacacaaccaggtgccgacggttttgaattgcagcacccgtatgtgctcgatgctggaatccctatatcacaattgacagtatgcccccatcaatggattaacctgcgcaccaataactgtgccacaataatagtgccatatatgaatacactgccttttgactccgccctgaaccattgcaattttggcctgttggtagtacccattagcccattggattttgaccaaggcgcaactccggttatccctattacaatcactcttgctccaatgtgctctgagtttgctggtctcaggcaggcagtcactcaaggctttcccactgagccaaaaccaggcacgaaccaattcttgaccaccgatgatggcgtctcagcacccattctgccaaatttccatcccactccatgtattcacatacccggtgaagtcagaaacctgctcgagttgtgtcaagtggagactattcttgaggttaacaacgtacccaccaatgctactagtttgatggaaagacttcgcttcccggtgtccgcgcaagcgggcaaaggtgaattgtgtgctgtgtttagggctgatcctggaagagacggtccatggcagtcaacgatgctgggccagttgtgtggatactacactcagtggtcaggctcactggaggtcactttcatgttcaccggctctttcatggccacgggtaagatgctcatagcttatacacctcctggcggtcccttgcccaaagatcgcgctacagcaatgctgggcacacatgttatttgggattttggccttcaatcatctgtcacccttgtgataccatggatcagtaatacccactacagagcgcatgccagggatggagtgttcgactactacaccacaggactggtcagtatctggtaccaaacaaattacgtagttccaattggcgcacccaacacagcttacataatagcactggcggcggcccagaagaatttcaccatgaaactgtgcaaagacactagtcacatattgcagacagcctctattcagggagatagggtggcagatgtgatcgagagctccatcgacaacaccgtgagggaaactgtcggcgcttcaaccagtagagatgcactgccaaatactgaatcctctggacccgcacattctaaggagatccctgcactgaccgccgtggaaactggcgccacaaaccccctggtgcctagcgacaccgtccagactagacacgtgatccagcatcggagccgctccgagtcaagtgtggaatctttctttgctcgtggagcatgcgtcacaatcatgaccgtggacaactctgcttcaacaacctctaaggataaactgttctcagtgtggaagatcacatacaaagacaccgtccagctcaggcgtaagctggagttctttacctattcccgctttgatatggaatttactttcgtgatcacagctaacttcaccgagactaacaatggtcacgcactgaatcaggtctaccagattatgtatgtgccccctggcgcccctgtcccagaaaagtgggacgattacacttggcagacaagctccaacccctctatcttctacacctatggcactgctcccgcaagaatctcagtgccttatgtcggaatttccaatgcttactctcacttttatgacggtttcagcaaggtgcctctgaaagaccagagtgccgagctgggtgattccctgtacggcgccgcttccctcaacgacttcggaatcctggccgtgcgtgtggtcaacgatcacaatccaacaaaggtgacctccaaaatccgtgtctatctgaagccaaaacatattcgggtgtggtgtccacgtccaccccgggcagtcgcttactatggtcccggcgtggactataaggatggtactctcgcacctctgagcactaaggatctgactacctattaagcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaatgggtccaagtcttgactttgctctttccctgctgagaaggaacatcaggcaagtccaaacagatcagggccattttaccatgttgggtgtcagggatcgcttggctgttctcccaaggcacgcacagcccggcaagactatttgggtggagcacaaacttgtgaacgtcctcgacgcaatcgagctggtggatgaacagggcgttaatttggaactcacactggtgacacttgacactaatgaaaaatttagagatatcaccaagttcattccagagaccattagtggcgctagtgatgcaactttggtgatcaatacagaacatatgccatcaatgtttgtcccagtgggcgacgtcgtgcagtatggattcttgaaccttagtggaaagccaacacataggaccatgatgtacaatttccctacaaaagcaggacaatgtggaggtgtggtcacatccgtcggtaagatcgttggtattcacattggcggcaacggccgccaaggtttctgtgctggtttgaagaggagttacttcgcaagtgtgcagggtgagatccaatgggtgaaacctaacaaagaaactggtagactgaacatcaatggaccaactcgcactaagctggagcctagtgtgtttcatgaagtgtttgaaggcaataaggaaccagcagtcttgacaagtaaagaccctagactggaggtcgactttgagcaagccctgttttccaagtatgtgggcaatgttttgcatgagcccgatgaatacgtgactcaagctgccctccactatgcgaatcaactcaaacagttggacataaacactagcaagatgagcatggaggaagcgtgctatggcactgagaacctggaagcaattgatctctgtactagtgccggatatccatacagcgcccttggcatcaagaaaagagacattctcgaccccgtaaccagggatgtgtctaagatgaaattctatatggacaaatacggcctcgatctgccatactccacctacgtaaaggatgagcttcgctctctggataaaatcaagaaaggaaagtcacgcctgatagaggctagtagcttgaatgactctgtctacctcagaatgacttttggccacctttacgaggtatttcatgctaaccctggcactgtgaccggttccgcagtgggttgcaacccagacgtgttttggagtaaacttccgatccttctgcctggctcactctttgcctttgactactcaggatatgatgctagcctcagcccggtatggttcagggcccttgaaattgtgttgcgcgaaattggctatccagaagaggccgtgtcccttatagaaggaatcaaccacactcaccacgtgtaccgcaacaaaacatactgtgtacttggtggcatgccctcaggctgttctggtacttccatctttaactcaatgatcaacaacatcatcattagaaccctcttgatcaaaacctttaagggaatagacctggatgagttgaacatggtggcctatggcgacgatgtgttggctagttatccctttcctatagattgccttgagttggctaagactggcaaagagtatggtttgactatgacgcctgcagacaaatcaccctgtttcaatgaagtgacgtgggaaaatgctaccttccttaagagaggcttcttgccagaccaccaatttccattcttgattcaccctacgatgcccatgagagagatccatgagtccattcgctggactaaagacgcacgcaatacccaagaccacgtgcgctccctgtgtctgttggcatggcacaacggtaaggatgaatatgagaaatttgtgagtgcaattagatcagttccagttggaaaagcgttggccattcctaattttgagaatttgagaagaaattggctcgaattgttttaaatcgatttaattaatagcataaccccttggggcctctaaacgggtcttgaggggttttttggaattcacccagctttcttgtacaaagtggt

(19) United States
(12) Patent Application Publication
(10) Pub. No.: US 2019/0224304 A1
(43) Pub. Date: Jul. 25, 2019

(54) CHIMERIC ENTEROVIRUS VIRUS-LIKE PARTICLES

(71) Applicant: SENTINEXT THERAPEUTICS Sdn Bhd, PENANG (MY)

(72) Inventor: Mary Jane CARDOSA, KUALA LUMPUR (MY)

(21) Appl. No.: 16/339,772

(22) PCT Filed: Sep. 21, 2017
(86) PCT No.: PCT/MY2017/050059

§ 371 Date: Apr. 5, 2019

(30) Foreign Application Priority Data
Oct. 7, 2016 (MY) PI 2016703709

(51) Int. Cl. A61K 39/125; A61K 39/13; A61K 39/135

(52) U.S. Cl. CPC A61K 39/125; A61K 39/13; A61K 39/135; A61K 2039/5258; C12N 2770/32322; C12N 2770/32323; C12N 2770/32371; C12N 2770/32334

(57) ABSTRACT

The invention provides chimeric Enterovirus virus-like particles (VLPs) for protection and/or treatment against infection by more than one Enterovirus. More specifically, the present invention provides chimeric EV-A71 virus-like particles displaying CV-A16 VP1 polypeptides and at the same time maintaining important neutralizing antibody epitopes of EV-A71 itself. More specifically, the present invention provides chimeric CV-A16 virus-like particles displaying EV-A71 VP1 polypeptides. Thus, the present invention provides a bivalent vaccine comprising the chimeric virus-like particles which elicit an immune response and/or neutralizing antibody response to both EV-A71 and CVA-16 for the treatment of Hand, Foot and Mouth Disease.

### TECHNICAL FIELD OF THE INVENTION

This invention relates to chimeric Virus-Like Particles (VLPs) derived from Enterovirus and vaccines comprising such VLPs that elicit an immune response and/or protective neutralizing antibody response directed against more than one Enterovirus.

### BACKGROUND OF THE INVENTION

Enterovirus is a genus of viruses belonging to Picomavirus, family Picomaviridae. Enterovirus represents a genus of a large and diverse group of small RNA viruses characterized by a single positive-strand genomic RNA. All enteroviruses contain a genome of approximately 7,500 bases and are known to have a high mutation rate due to low-fidelity replication and frequent recombination. After infection of the host cell, the genome is translated in a cap-independent manner into a single polyprotein, which is subsequently processed by virus-encoded proteases into the structural capsid proteins and the nonstructural proteins, which are mainly involved in the replication of the virus.

The enteroviruses are associated with several mammalian diseases. Enteroviruses are classified into 12 species as follows: Enterovirus A, Enterovirus B, Enterovirus C, Enterovirus D, Enterovirus E, Enterovirus F, Enterovirus G, Enterovirus H, Enterovirus J, Rhinovirus A, Rhinovirus B and Rhinovirus C.

Within the twelve species of Enterovirus there are many serotypes. Serologic studies have distinguished numerous Enterovirus serotypes on the basis of antibody neutralization tests.

Species Enterovirus A includes, for example, serotypes EV-A71 (also referred to as EV71 or HEV71), EV-A76, EV-A89, EV-A90, EV-A91, EV-A92, CV-A16 (Coxsackievirus A16), CV-A5, CV-A6, and CV-A10.

Species Enterovirus C exhibits 23 serotypes, which include, for example, PV-1 (Poliovirus 1), PV-2, PV-3, CV-A20, CV-A21, EV-C95, EV-C96, EV-C99, EV-C102, EV-C104, EV-C105, and EV-C109.

Serotypes EV-D68, EV-D70, and EV-D94 are classified under the species Enterovirus D.

All members of the genus Enterovirus, including EV-A71, polioviruses and Coxsackievirus A16 have a single stranded positive sense RNA genome, which has a single open reading frame encoding a polyprotein, P1, consisting of the capsid structural proteins VP0, VP3 and VP1, and several non-structural proteins including the viral proteases 3C and 3CD which are responsible for cleaving the polyprotein P1 into the individual capsid proteins VP1, VP3 and VP0, which VP0 is eventually cleaved into VP2 and VP4 after viral RNA encapsidation. The capsid proteins, VP0, VP1 and VP3, may assemble into virus-like particles (VLPs) without encapsidation of the genome, but cleavage of VP0 into VP2 and VP4 occurs after RNA encapsidation during maturation of the native virus.

Diseases caused by enterovirus infection include poliomyelitis which is the most notable disease caused by an Enterovirus infection. Examples of other diseases are aseptic meningitis, hand, foot and mouth disease (HFMD), conjunctivitis, respiratory illnesses and myocarditis. Nonspecific febrile illness is, however, the most common presentation of an Enterovirus infection.

Infection with Enterovirus C and especially polioviruses have been a widespread problem, and epidemics of poliomyelitis have historically been a major global health problem causing millions of deaths during the 20^{th} century. Inactivated whole virus vaccines have been used for mass immunization and are currently available and used for prophylaxis against poliovirus infection. Good results, leading to eradication of poliomyelitis in most countries of the world, have been obtained with inactivated poliomyelitis vaccines, which may be prepared according to a method which has been developed by Jonas Edward Salk and has been improved later in several aspects. Generally, these vaccines contain a mixture of inactivated polio viruses of strains Mahoney, MEF1 and Saukett. Although attenuated poliovirus serotypes PV-1, PV-2 and PV-3 strains (Sabin) have been produced and used as an attenuated oral polio vaccine, the attenuated Sabin vaccine occasionally produces revertants leading to what has been referred to as Vaccine Associated Paralytic Polio (VAPP). Typically, vaccination with the individual polypeptides of polioviruses in the form of a subunit vaccine has shown that the isolated polypeptides are not capable of raising neutralizing antibodies in humans and animals (Meloen. et al., J. Gen. Virol. 45:761-763, 1979).

Otherwise, enteroviruses are the most common causes of aseptic meningitis in children. In the United States, enteroviruses are responsible for 30,000 to 50,000 cases of meningitis. Encephalitis is a rare manifestation of an enterovirus infection; but when it occurs, the most frequent enterovirus found to be causing the encephalitis is echovirus 9. Pleurodynia caused by enteroviruses is characterized by severe paroxysmal pain in the chest and abdomen, along with fever, and sometimes nausea, headache, and emesis. Pericarditis and/or myocarditis are typically caused by enteroviruses. Arrythmias, heart failure, and myocardial infarction have also been reported. Acute hemorrhagic conjunctivitis can be caused by enteroviruses.

Enterovirus infection may cause hand, foot and mouth disease (HFMD). HFMD is a common, self-limiting childhood illness most commonly caused by infection by Coxsackievirus A16 (CV-A16) virus or EV-A71, but also other Enterovirus A serotypes such as CV-A2, CV-A4, CV-A5, CV-A6, CV-A7 and CV-A10, may cause hand, foot and mouth disease, and in addition CV-B1, CV-B2 and CV-B5 may cause HFMD (Li Y, Zhu R, Qian Y, Deng J (2012) The Characteristics of Blood Glucose and WBC Counts in Peripheral Blood of Cases of Hand Foot and Mouth Disease in China: A Systematic Review. PLoS ONE 7(1): e29003; published Jan. 3, 2012).

However, Enterovirus 71 (EV-A71) and Coxsackievirus A16 (CV-A16) are the Enterovirus serotypes notable as the major causative agents for HFMD, but in addition EV-A71 is frequently also associated with severe central nervous system complications and in some cases cardiovascular system manifestations. EV-A71 was first isolated and characterized from cases of neurological disease in California in 1969. To date, little is known about the molecular mechanisms of host response to EV-A71 infection, but increases in the level of mRNAs encoding chemokines, proteins involved in protein degradation, complement proteins, and pro-apoptotic proteins have been implicated.

Over the past decade and a half, HFMD has emerged as a worldwide public health problem particularly in the Asia-Pacific region wherein the disease is caused by a group of non-polio enteroviruses of the Picomaviridae family of which Coxsackievirus A16 (CV-A16) and Enterovirus 71 (EV-A71) are the most common etiological agents. Fatal EV-A71 were first seen in Sarawak, Malaysia in 1997 followed by a large outbreak in Taiwan in 1998 and then annually in one or another country in the Asia Pacific. A huge EV-A71 outbreak was seen in China in 2008 and this disease was made notifiable in China and other countries. According to the World Health Organization (WHO) situational update (dated 11 Dec. 2013), for the year of 2013 there were 1,651,959 cases with 265 deaths reported in China, 71,627 cases with 19 deaths reported in Vietnam and 294,535 cases reported in Japan amongst other countries reflecting the profound extent of the HFMD disease according to the WHO. In May 2013, 5 cases of polio-like paralysis (1 case in Victoria) with 27 severe EV-A71-associated cases were reported in New South Wales, Australia as a result of an EV-A71 strain that has been circulating in Asia for some time and was only detected recently in Australia, implicating global travel playing a key role in facilitating disease transmission. In China where the majority of HFMD cases are seen, there continues to be regular outbreaks peaking in May and June from 2009 through 2015.

In HFMD, enteroviruses are excreted in feces and are also found in pharyngeal secretions. Transmission is associated with close contact among children and through environmental contamination. Disease is characterized by an acute onset of fever with a rash on the palms, soles, buttocks, and knees, and vesicles on buccal membranes that usually resolve in 7-10 days. However, a small proportion of children with HFMD develop severe central nervous system disease which is often fatal.

Severe HFMD disease involving primarily the neurologic and cardiovascular systems manifesting as syndromes such as meningitis, encephalitis, acute flaccid paralysis, pulmonary edema and cardiac failure generally occur only with EV-A71 infection. In the Asia-Pacific Region the most devastating neurological syndrome is brainstem encephalitis, which has a mortality rate of 40-80 percent. Children with severe HFMD may take months to recover, and in some cases the neurologic damage may be permanent. Currently, there is no specific antiviral treatment for HFMD.

With regard to vaccine development, there are numerous publications describing approaches to prepare novel vaccines directed against Enterovirus A serotypes such as EV-A71 and CV-A16, and especially against EV-A71. Three companies in China, as the country most affected by large EV-AV71 outbreaks, have completed phase III trials of an inactivated whole virus EV-A71 monovalent vaccine. The major drawback with such vaccines is risk of infection due to incomplete inactivation, as well as environmental risks during production.

Subunit protein vaccines based especially upon the EV-A71 VP1 protein have been evaluated in academic settings without progressing to more commercial development. A good example is from WU et al., (Vaccine 20, 895-904 (2002)) where a VP1 subunit vaccine was immunogenic and elicited neutralizing antibodies, but was inferior to an inactivated whole virus vaccine control in both titre and duration of effect, with in vivo protection only seen with low titre virus challenge and not a high titre virus challenge.

In addition, more advanced state-of-the-art technologies have been used to develop prototype vaccines and vaccines in early preclinical stages for both EV-A71 and CV-A16.

For example, virus-like particles of Enterovirus EV-A71 are described by CHUNG et al. (2006) (World J Gastroenterol 12(6): 921-927, 2006), CHUNG et al. (2008) (Vaccine 26:1855-1862, 2008), and CHUNG et al. (2010) (Vaccine 28:6951-6957, 2010) disclosing an EV-A71 VLP consisting of EV-A71 structural polypeptides VP0, VP1 and VP3.

Furthermore, the present applicant's earlier published patent application; International Publication Number WO 2013/098655 discloses EV-A71, CV-A16 and poliovirus VLPs comprising VP0 polypeptides, VP1 polypeptides, VP2 polypeptides, VP3 polypeptides and VP4 polypeptides.

Since both EV-A71 and CV-A16 co-circulate and cause HFMD with approximately equal frequency in South East Asia, vaccination against both pathogens is required for protection against clinical HFMD diseases. Phase III trials already conducted in China with the monovalent inactivated whole virus EV-A71 vaccines show very clearly that there is no cross protection against non EV-A71 enteroviruses as shown in ZHU. et al. (The Lancet, 381:2024-2032, 2013); ZHU, et al. (NEJM 370(9):818-828, 2014) and LI et al. (New England Journal of Medicine 370(9):829-837, 2014).

This lack of cross protection between different enterovirus serotypes presents a problem for public health, particularly in the field of HFMD. Since there are several Enterovirus A serotypes that cause clinical HFMD but only EV-A71 causes significant neurological disease and death, treatment with an EV-A71 mono-protective vaccine will only protect against EV-A71 infection.

Efficacious vaccines for protection against EV-A71 and other enteroviruses are strongly needed and, optimally, this should be in the form of bivalent vaccines protecting as a minimum against infection by both EV-A71 and CV-A16 to provide complete protection against HFMD.

In addition, there is a strong need for vaccines against enteroviruses causing neurological disease, and such vaccine optimally would provide protection against both EV-A71 and polioviruses.

An ideal vaccine against hand, foot and mouth disease (HFMD) should at least be bivalent against both EV-A71 and CV-A16. Several strategies for preparing such vaccines and vaccine formulations have been researched.

However, there are many challenges to overcome in the development of bivalent or multivalent vaccines. A commercial vaccine formulation comprising simple mixtures of monovalent Enterovirus vaccines such as for EV-A71 and CV-A16 should be possible, but it is unknown whether immune dominance and interference between the vaccine components may be problematic GONG, et al. (Journal of Virology 88(11): 6444-6452, 2014). In addition, such an approach carries the vaccine development and manufacturing costs for two or multiple vaccines, and may be prohibitive for development and sale in many regions of the world where EV-A71 and CV-A16 are health risk factors.

Nonetheless, K U, et al. discloses: “The primary aim of this study is thus to determine whether a bivalent vaccine formulation comprised of both EV71- and CA16-VLPs can induce a balanced protective immune response. Our results show that bivalent VLP immunization elicited neutralizing antibody response to each of the viruses were at levels comparable to that induced by corresponding monovalent VLP, indicating the absence of interference between the two VLPs with respect to their ability to induce virus-specific neutralizing antibodies”, indicating, that with the actual vaccine constructs, the actual vaccine composition and in the specific mouse experiment, interference and immune dominance between the two VLPs with respect to their ability to induce virus-specific neutralizing antibodies is absent, contrary to experience with trivalent inactivated polio vaccines in humans (KU, et al., Vaccine 32(34):4296-303, 2014).

With more sophisticated technologies mutations have been introduced into EV-A71 VLP vaccines to introduce bivalency. ZHAO, et al. (Scientific Reports 5:7878, 19 Jan. 2015, DOI: 10.1038/srep07878) disclose a mutated EV-A71 VLP, wherein in the SP70 amino acid sequence of the EV-A71 VP1 is replaced with the homologous SP70 amino acid sequence (4 of 15 amino acids different) from CV-A16 VP1 in an EV-A71 VLP. While immunization with the mutated VLPs induced robust neutralizing antibody responses against EV-A71 similar to that of EV-A71 VLPs, and provided full protection to neonatal mice against lethal EV-A71 challenge, the neutralizing antibody titers induced by the mutated or “chimeric” VLPs was only 1:16 against CV-A16. The anti-CV-A16 neutralizing antibody titer is well-below that which was previously observed using an inactivated CV-A16 vaccine which was reported to elicit neutralizing antibody titers of 1:256 and a CV-A16 VLP vaccine which was reported to elicit neutralizing antibody titers of about 1:128. Thus, based on the low levels of neutralizing antibody titers induced by the mutated VLPs against CV-A16, it can be concluded that the efficacy of a single epitope for protecting against infection of viruses is limited.

The above example of a mutated or so-called “chimeric” vaccine uses linear epitopes and inserts these into a carrier that may be EV-A71 itself. One problem with this approach is the efficacy of the peptide or peptides chosen to be delivered in this manner. For the most part the results from the peptides used are not always reproducible by different groups and the neutralizing antibody titres generated are low as described in two review papers by CHONG, et al. (Vaccines & Immunotherapeutics, 2012, 8:12, 1775-1783, DOI: 10.4161/hv.21739; and CHOU, et al. (Clinical and Developmental Immunology, 2012, DOI:10.1155/2012/831282).

Picomavirus capsid structure is characterized by a five-fold vertex surrounded by a canyon in which a pocket factor is located which stabilizes the capsid structure. When the virus interacts with its cellular receptor, the binding of the receptor is often to a wall of the canyon, displacing the pocket factor and causing a structural change causing the virus to form what is called the A particle wherein there are holes formed which allow the virus genome to escape into the cell. ROSSMANN, et al. (Trends in Microbiology (2002) vol 10 No 7 324-331) reviews the Picornavirus-receptor interactions very well. Thus, an antibody which blocks the interaction of the capsid to the virus receptor will function as a neutralizing antibody and block infection.

EV-A71 has neutralizing epitopes which are located elsewhere and not on the five-fold vertex as shown in PLEVKA, et al. (Proc. Natl. Acad. Sci. 111(6):2134-9 (2014)). There are neutralizing epitopes on the two-fold and three-fold axes involving interactions between VP3 and VP2.

Enterovirus VLPs comprising the structural polypeptide, VP0, VP1 and VP3, may be produced from a cassette encoding an Enterovirus P1 polypeptide and Enterovirus 3C and/or 3CD proteases (CHUNG, et al., World J Gastroenterol 12(6):921-927, 2006).

It was shown by applicants earlier published patent application; International Publication Number WO 2013/098655 that VLPs may be formed comprising enterovirus VP0 polypeptides, VP1 polypeptides, VP2 polypeptides, VP3 polypeptides and VP4 polypeptides. Moreover, it was shown that the VP2 protein therein also harbors neutralizing antibody epitopes.

It has never been shown that a chimeric capsid or a chimeric VLP can be formed wherein VP1 polypeptides from one Enterovirus and VP0 polypeptides and VP3 polypeptides and/or in addition VP2 polypeptides and VP4 polypeptides from a different Enterovirus self-assemble into a VLP.

It has never been shown that such chimeric capsid or a chimeric VLP may exhibit neutralizing antibody epitopes against any one or both of the Enteroviruses from which the VP1, the VP0 and VP3 and/or in addition VP2 and VP4 polypeptides originate.

Moreover, it has never been shown that such capsid or a chimeric VLP as a vaccine may elicit protective and/or neutralizing immune responses against both of the Enteroviruses from which the VP1, VP0 and VP3 and/or in addition VP2 and VP4 originate.

It is indeed well recognized in the vaccine art, that it is unclear whether an antigen derived from a pathogen will elicit protective immunity. ELLIS (Chapter 29 of Vaccines, PLOTKIN, et al. (eds) WB Saunders, Philadelphia, at page 571, 1998) exemplifies this problem in the recitation that “the key to the problem (of vaccine development) is the identification of that protein component of a virus or microbial pathogen that itself can elicit the production of protective antibodies, and thus protect the host against attack by the pathogen.” With the instant invention, we have achieved viable and protective VLPs which are shown to be protective against multiple members of the genus Enterovirus.

### BRIEF SUMMARY OF THE INVENTION

A chimeric Virus-Like Particle (VLP) assembled from Enterovirus polypeptides VP0 and VP3 and a heterologous VP1 polypeptide, wherein the Enterovirus is selected from Enterovirus A, Enterovirus B, Enterovirus C and Enterovirus D, such a

chimeric VLP additionally assembled from Enterovirus polypeptides VP2 and/or VP4, such a

chimeric VLP wherein the polypeptides VP0 and VP3 are derived from Enterovirus EV-A71 and the heterologous Enterovirus VP1 polypeptide is from Enterovirus A, Enterovirus B, Enterovirus C or Enterovirus D, such a

chimeric VLP wherein the heterologous Enterovirus VP1 polypeptide is from Enterovirus CV-A16, such a

chimeric VLP wherein the heterologous Enterovirus VP1 polypeptide is from Enterovirus C selected from Poliovirus 1, Poliovirus 2 and Poliovirus 3, such a

chimeric VLP wherein the polypeptides VP0 and VP3 are derived from Enterovirus CV-A16 and the heterologous Enterovirus VP1 polypeptide is from Enterovirus A, Enterovirus B, Enterovirus C or Enterovirus D, such a

chimeric VLP wherein the heterologous Enterovirus VP1 polypeptide is from Enterovirus EV-A71, such a

vaccine comprising chimeric VLPs assembled from Enterovirus EV-A71 VP0 and VP3 polypeptides and a heterologous VP1 polypeptide of an Enterovirus selected from Enterovirus A, Enterovirus B, Enterovirus C and Enterovirus D, such a

vaccine wherein the chimeric VLPs are additionally assembled from Enterovirus EV-A71 VP2 and/or VP4 polypeptides, such a

vaccine wherein the chimeric heterologous Enterovirus VP1 polypeptide is from Enterovirus A, such a

vaccine wherein the Enterovirus A is CV-A16, such a

vaccine wherein the heterologous Enterovirus VP1 polypeptide is from an Enterovirus C selected from Poliovirus 1, Poliovirus 2 and Poliovirus 3, such a

vaccine comprising chimeric VLPs assembled from Enterovirus CV-A16 VP0 and VP3 polypeptides and a heterologous VP1 polypeptide of an Enterovirus selected from Enterovirus A, Enterovirus B, Enterovirus C and Enterovirus D, such a

vaccine wherein the chimeric VLPs are additionally assembled from Enterovirus CV-A16 VP2 and/or VP4 polypeptides, such a

vaccine wherein the chimeric heterologous Enterovirus VP1 polypeptide is from Enterovirus A, such a

vaccine wherein the Enterovirus A is EV-A71, such a

vaccine including one or more vaccine adjuvants, such a

vaccine wherein the adjuvant(s) is selected from ISCOMS, alum, aluminum hydroxide, aluminum phosphate, Quil A and other saponins, such a

chimeric VLP for use in a vaccine for vaccinating a subject against infection by more than one Enterovirus, the use comprising administering to the subject the chimeric VLP in an amount effective to elicit a protective and/or neutralizing immune response to the more than one Enterovirus when administered to the subject, such a

method of providing an immune response and/or neutralizing immune response against infection by more than one Enterovirus in a subject, the method comprising administering to the subject a chimeric VLP in an amount effective to provide such immune response and/or neutralizing immune response, such a

nucleic acid encoding an expression cassette, the expression cassette comprising a promoter operably linked to a nucleic acid encoding a chimeric Enterovirus polypeptide P1, the chimeric polypeptide P1 comprising Enterovirus structural polypeptides VP0 and VP3 and a heterologous VP1 structural polypeptide of an Enterovirus selected from Enterovirus A, Enterovirus B, Enterovirus C or Enterovirus D, wherein the nucleic acid encoding the chimeric Enterovirus polypeptide P1 is operably linked to an nucleic acid encoding an Internal Ribosome Entry Site (IRES) and an Enterovirus 3CD protease, wherein the 3CD protease is under the translational control of the IRES, such a

expression cassette wherein the Enterovirus structural polypeptides VP0 and VP3 are from Enterovirus EV-A71, such a

expression cassette wherein the heterologous VP1 structural polypeptide is from Enterovirus CV-A16, such a

expression cassette wherein the Enterovirus structural polypeptides VP0 and VP3 are from Enterovirus CV-A16, such a

expression cassette wherein the heterologous VP1 structural polypeptide is from Enterovirus EV-A71, such a

expression cassette wherein the nucleic acid sequence encoding the IRES is from Encephalomyocarditis virus (EMCV), such a

expression cassette wherein the nucleic acid sequence encoding the IRES has been genetically modified, such a

expression cassette wherein the nucleic acid sequence encoding the 3CD has been genetically modified, such a

expression cassette wherein the IRES is derived from Encephalomyocarditis virus (EMCV) or an Enterovirus, such a

method of making a chimeric Enterovirus VLP comprising the step of culturing a host cell comprising an expression cassette for a period of time sufficient to produce the chimeric Enterovirus polypeptide P1 and Enterovirus 3C or 3CD proteases, and to form VLPs, such a

method further comprising the step of recovering the VLPs from the host cell, such a

method wherein the host cell is a eukaryotic cell.

### BRIEF DESCRIPTION OF THE FIGURES

FIG. 1. A. Depiction of the arrangement of structural capsid proteins of a native P1 polypeptide. B. Depiction of the arrangement of structural capsid proteins of a chimeric P1 polypeptide, wherein the VP1 polypeptide is from an Enterovirus which is different than the Enterovirus from which the VP0 and VP3 polypeptides derive.

FIG. 2. Chimeric Enterovirus expression cassettes [chimeric P1+IRES+3CD]. A. Chimeric expression construct SXT8, the expression cassette encoding EV-A71 VP0 and VP3 polypeptides and a CV-A16 VP1 polypeptide. B. Chimeric expression construct SXT7, the expression cassette encoding CV-A16 VP0 and VP3 polypeptides and an EV-A71 VP1 polypeptide.

FIG. 3. Western blot of lysates from cells which were infected with baculovirus expression constructs. The Western blot was probed with a rabbit polyclonal antibody directed against an EV-A71 VP1 polypeptide. The VP1 polypeptide is indicated by the arrow.

FIG. 4. Western blot of lysates from cells which were infected with baculovirus expression constructs. The Western blot was probed with the mouse monoclonal antibody F6/2/A1-1/2/A3 directed against a CV-A16 VP1 polypeptide. The VP1 polypeptide is indicated by the arrow.

FIG. 5. ELISA plates coated with recombinant subunit EV-A71 capsid protein VP1 (Panel A) or EV-A71 capsid protein VP0 (Panel B) were used to react with sera obtained from mice immunized with the VLPs from the chimeric SXT7 or with sera obtained from mice immunized with VLPs from the non-chimeric SN07 having a native EV-A71 P1 polypeptide. SXT7 VLPs have VP1 polypeptides which derive from Enterovirus EV-A71. The graph shows that both the sera from mice immunized with SXT7 VLPs, as well as the non-chimeric EV-A71 VLPs, bind to the VP1 protein of EV-A71 (Panel A). The sera from the mice immunized with the non chimeric EV-A71 VLP, but not sera from mice immunized with the SXT7 VLPs, bind to EV-A71 VP0 (Panel B).

FIG. 6. Sandwich ELISA detecting the presence of various monoclonal antibody epitopes on VLPs. E18 and E19 are monoclonal antibodies specific for EV-A71 quarternary epitopes on native Enterovirus EV-A71 virus. The Y axis shows optical density at a 450 nm wavelength (OD₄₅₀) As shown by the positive signals, quarternary epitopes of monoclonal antibodies EV18 and EV19 are present on the SXT8 VLPs indicating that there is proper assembly of the capsid proteins having intact EV-A71 specific neutralizing epitopes.

FIG. 7. Indirect ELISA using sera from mice immunized with SXT8 VLPs. ELISA plates coated with recombinant subunit EV-A71 capsid polypeptide VP1 (Panel A) or EV-A71 capsid polypeptide VP0 (Panel B) were used to react with sera from mice immunized with the VLPs from the chimeric SXT8 or sera from mice immunized with the VLPs from non-chimeric SN07 having a native EV-A71 P1 polypeptide.

FIG. 8. The presence of antibodies against EV-A71 and CV-A16 in sera from animals immunized with VLPs from chimeric SXT7, VLPs from chimeric SXT8, or with the control antigen, FGUS. Wells were coated with lysates from mock-infected rhadbomyosarcoma (RD) cells, CV-A16-infected RD cells (Panel A), or EV-A71-infected RD cells (Panel B). The net OD at 450 nm was calculated by subtracting the OD values of wells coated with lysates from virus-infected RD cells with that of mock-infected RD cells.

FIG. 9. Enterovirus expression cassettes comprising poliovirus PV1 structural polypeptides. A. Expression construct SXT11, the expression cassette encoding a poliovirus PV1 native P1 polypeptide, an IRES, and an EV-A71 3CD protease. B. Chimeric expression construct SXT18, the expression cassette encoding EV-A71 VP0 and VP3 polypeptides and a poliovirus PV1 VP1 polypeptide, an IRES, and an EV-A71 3CD protease.

FIG. 10. EV-A71 VLP expression cassette [P1+IRES+3CD] in the pSN01 baculovirus expression construct which gives rise to the baculovirus expression construct, SN07, described in PCT/IB2012/003114 comprising an Enterovirus EV-A71 P1, an IRES and an EV-A71 3CD protease.

### DETAILED DESCRIPTION OF THE INVENTION

The invention provides chimeric virus-like particles (VLPs) for protection and/or treatment against infection by more than one Enterovirus. The invention further provides chimeric virus-like particles (VLPs) in the form of an immunogenic composition and/or vaccine for protection and/or treatment against infection by more than one Enterovirus. More specifically, the present invention provides chimeric EV-A71 virus-like particles displaying CV-A16 VP1 polypeptides/epitopes and at the same time maintaining important neutralizing antibody epitopes of EV-A71 itself. Thus, the present invention provides a vaccine comprising a chimeric virus-like particle which elicits immune responses and/or neutralizing antibody responses to both EV-A71 and a different Enterovirus selected from those members of Enterovirus A, Enterovirus B, Enterovirus C or Enterovirus D and any serotype virus of these virus species.

More specifically the present invention provides a vaccine comprising a chimeric virus-like particle which elicits immune responses and/or neutralizing antibody responses directed against more than one Enterovirus comprising one or more epitopes of VP0, VP2, VP3 and/or VP4 of EV-A71 and one or more epitopes of VP1 of a different enterovirus selected from Enterovirus A, Enterovirus B, Enterovirus C or Enterovirus D and any serotype virus of these virus species. It has surprisingly been found that such chimeric VLPs actually can assemble to form stable virus-like particles. More importantly, such virus-like particles exhibit one or more epitopes of VP0, VP2, VP3 and/or VP4 of EV-A71 and one or more epitopes of VP1 of a different enterovirus selected from Enterovirus A, Enterovirus B, Enterovirus C or Enterovirus D and any serotype virus of these virus species. Such chimeric VLPs, when administered in an effective dose, elicit immune responses and/or neutralizing antibody responses against both an Enterovirus infection caused by the species and serotype of virus from which the VP1 originates as well as against EV-A71.

The invention therefore provides vaccines and vaccine formulations comprising virus-like particles including EV-A71 polypeptides VP0 and VP3, and optionally enterovirus polypeptides EV-A71 VP2 and/or VP4, and heterologous VP1 polypeptides of an Enterovirus selected from Enterovirus A, Enterovirus B, Enterovirus C or Enterovirus D. Such chimeric virus-like particles are shown to provide protection and/or neutralizing antibody responses against more than one Enterovirus. For clarification, such immune responses and/or neutralizing antibody responses are directed against more than one Enterovirus serotype, which response is distinct from immune responses and/or neutralizing antibody responses against different strains within the same serotype.

Accordingly, the VLPs of the present invention may comprise VP1 polypeptides of an Enterovirus selected from Enterovirus A, Enterovirus B, Enterovirus C or Enterovirus D, which VP1 polypeptides may comprise the complete VP1 sequence of said Enterovirus. The VP1 polypeptides may comprise the complete VP1 sequence of said Enterovirus selected from Enterovirus A, Enterovirus B, Enterovirus C or Enterovirus D and in addition up to 50 amino acids from the flanking C-terminal portion of a VP3 polypeptide from a heterologous Enterovirus. Alternatively, the VP1 polypeptides may be truncated VP1 polypeptides of said Enterovirus selected from Enterovirus A, Enterovirus B, Enterovirus C or Enterovirus D, comprising in addition amino acids of a VP1 polypeptide from a heterologous Enterovirus corresponding and homologous to those deleted amino acids of the VP1 polypeptide of said Enterovirus.

In an embodiment, the chimeric VLPs comprise Enterovirus EV-A71 VP0 and VP3 polypeptides, and Enterovirus CV-A16 VP1 polypeptides.

In a further embodiment, the chimeric VLPs comprise Enterovirus CV-A16 VP0 and VP3 polypeptides, and Enterovirus EV-A71 VP1 polypeptides.

In an additional embodiment, the chimeric VLPs comprise Enterovirus EV-A71 VP0 and VP3 polypeptides, and Enterovirus Poliovirus-1 (PV1) VP1 polypeptides.

The invention in an additional aspect includes a method for production of the chimeric VLPs, which method may include the steps of: (i) constructing an expression cassette comprising a promoter operably linked to a nucleic acid which encodes a chimeric Enterovirus P1 polypeptide, which nucleic acid is operably linked to an internal ribosome entry site (IRES), which IRES nucleic acid is also operably linked to a nucleic acid encoding an Enterovirus 3C or 3CD protease; (ii) transfecting, transforming or infecting a suitable host cell with a construct containing the expression cassette; (iii) culturing the host cells under conditions in which chimeric virus like particles (VLPs) are produced by the cell after expression of the nucleic acids comprised in the cassette.

Making truncations and mutations of the 3CD protease in the expression cassette may achieve increased yield of VLPs. For example, the Glycine of the EV-A71 3C protease, which is amino acid 1671 of GenBank accession number DQ341362.1 may advantageously be changed to an Alanine (G1671A) using site directed mutagenesis for the expression of mutant EV-A71 3C and subsequent processing of an Enterovirus P1 polypeptide.

Expression cassettes cloned into vectors, such as for example baculovirus vectors, and transformed, transfected or infected into appropriate prokaryotic or eukaryotic host cells, such as for example insect cells, such as but not limited to Spodoptera frugiperda (e.g. Sf9 cells), for expression and purification of the VLPs of the invention are provided.

Chimeric P1 polypeptides were constructed, wherein structural polypeptides VP0 and VP3 comprised in the P1 polypeptide are from one Enterovirus species or serotype, and VP1 structural polypeptides are derived from a heterologous Enterovirus species or serotype.

In an embodiment, chimeric P1 polypeptides were constructed wherein the VP0 and VP3 structural polypeptides originate from Enterovirus EV-A71 and the VP1 structural polypeptides originate from Poliovirus (PV).

In an further embodiment, chimeric P1 polypeptides were constructed wherein the VP0 and VP3 structural polypeptides originate from Enterovirus EV-A71 and the VP1 structural polypeptides originate from CV-A16.

In another embodiment, chimeric P1 polypeptides were constructed wherein the VP0 and VP3 structural polypeptides originate from Enterovirus CV-A16 and the VP1 structural polypeptides originate from EV-A71.

The chimeric VLPs which are produced from the expression cassettes exhibit structural capsid polypeptides, which indicates that the chimeric P1 polypeptides have been processed by the EV-A71 3CD protease, and that the structural capsid polypeptides are assembled into VLPs.

The chimeric VLPs of the invention were analyzed using EV18 and EV19 which are EV-A71-specific monoclonal antibodies which recognize epitopes formed from the proper assembly of VP0/2 and VP3 polypeptides of native EV-A71 virus. The binding footprints of these monoclonal antibodies have been described in the publication PLEVKA, et al. The chimeric VLPs of the invention were also analyzed using Mab979 (Merck Millipore). MAb979 is a commercially available monoclonal antibody which recognizes a linear epitope in the Enterovirus VP2 structural polypeptide.

It is shown that quarternary epitopes of monoclonal antibodies EV18 and EV19 are present on chimeric SXT8 VLPs indicating that there is proper assembly of the capsid proteins, the chimeric VLPs having intact EV-A71 specific VP0/2 and VP3 neutralizing epitopes.

Thus, the chimeric VLPs contain assembled particles wherein the VP0/2 and VP3 polypeptides of the chimeric VLPs are intact and functional despite the fact that VP1 structural polypeptides in these chimeric VLPs are from CV-A16 and not EV-A71.

Moreover, it is demonstrated that serum antibodies from animals immunized against chimeric SXT8 VLPs (containing CV-A16 VP1 structural polypeptides) recognize CV-A16 VP1 polypeptides.

The results shown in Table 1 demonstrate that the chimeric SXT8 VLPs which consist of EV-A71 VP0/2 and VP3 structural polypeptides, and VP1 structural polypeptides from CV-A16 elicit a strong immune response directed against structural polypeptides from both EV-A71 and CV-A16.

TABLE 1

| | CV-A16 | CV-A16 | EV-A71 | EV-A71 |
|---|---|---|---|---|
| | VP0 | VP1 | VP0 | VP1 |
| SXT8 | <500 | 4000 | 8000 | 500 |
| serum | | | | |
| antibodies | | | | |

It is demonstrated that the important functional epitopes of EV-A71 are preserved, showing that the VLPs are assembled correctly, and the heterologous VP1 polypeptides from CV-A16 are also intact, immunogenically displayed on the VLPs and also elicit strong antibody responses.

Furthermore, the chimeric VLPs of the invention are demonstrated to elicit protective and/or neutralizing antibody responses against both Enterovirus EV-A71 and CV-A16.

Thus, a bivalent vaccine may be achieved utilizing a single immunogen consisting of the chimeric VLPs of this invention. Such chimeric VLPs provide epitopes eliciting bivalent immune responses which are protective against infection by both Enterovirus EV-A71 and CV-A16.

The chimeric VLPs provided by this invention thus enable the achievement of a bivalent Enterovirus vaccine utilizing a single chimeric VLP, thus making such vaccine much more convenient. It is thus easier and cheaper to produce a bivalent Enterovirus vaccine without having to produce, purify and mix multiple disparage VLPs, subunits, or antigens together to achieve bivalency.

### Pharmaceutical Formulations:

Pharmaceutically useful compositions comprising the chimeric VLPs of the invention may be formulated according to known methods such as by the admixture of pharmaceutically and immunologically acceptable carriers and/or adjuvants and/or additional antigenic determinants. Examples of such carriers and methods of formulation may be found in Remington's Pharmaceutical Sciences. To form a pharmaceutically acceptable composition suitable for effective administration, such compositions will contain an effective amount of one or more of the VLPs of the invention. Such compositions may contain VLPs derived from more than one type of Enterovirus.

Vaccine compositions of the invention may be administered to an individual in amounts sufficient to elicit immune responses and/or neutralizing antibody responses directed against more than one Enterovirus. The effective amount may vary according to a variety of factors such as the individual's condition, weight, sex and age. Other factors include the mode of administration. The vaccines may be provided to the individual by a variety of routes such as subcutaneous, topical, oral, mucosal, intravenous, parenterally, and intramuscular.

The vaccines comprising one or more of the chimeric VLPs of the invention may contain additional antigenic determinants and/or adjuvants well known in the art to elicit immune responses and/or neutralizing antibody responses in the host. Such vaccines are generally safe and do not have toxic side effects, may be administered by an effective route, are stable, and are compatible with vaccine carriers known in the art.

The vaccine may be administered in dosage forms known in the art such as for example, but not limited to, a form for injection, capsules, suspensions, elixirs, or liquid solutions. The vaccine may be administered in single or multiple doses. The invention in another aspect includes one or more of the VLPs of the invention in combination with one or more suitable adjuvants such as ISCOMS, alum, aluminum hydroxide, aluminum phosphate, Quil A and other saponins or any other adjuvant as described, for example, in VANSELOW (1987) S. Vet. Bull. 57 881-896. The meaning of the terms “aluminum phosphate” and “aluminum hydroxide” as used herein includes all forms of aluminum phosphate or aluminum hydroxide, which are suitable for adjuvanting vaccines.

### Definitions

Bivalent: Bivalent when used to describe a VLP means that the VLP will elicit an immune response directed against two members of the genus Enterovirus.

Chimeric: Chimeric, when used to describe a VLP, means a VLP wherein structural polypeptides or capsid proteins of one Enterovirus have been replaced with corresponding structural polypeptides or capsid proteins from a heterologous Enterovirus. A chimeric VLP does not mean VLPs with amino acid mutation or mutations in one or more epitopes.

Heterologous Enterovirus: Heterologous Enteroviruses are two or more Enteroviruses which belong to different families, species, serotypes, genogroups or strains.

Neutralizing immune response: A neutralizing immune response is an immune response wherein specialized cells of the immune system recognize the presentation of antigen(s), and launch specific immune responses, which prevent infection from an agent, for example a virus.

In an embodiment, the VLPs according to the invention can induce immune responses. The term “immune response” and/or “neutralizing antibody response” as used herein is intended to mean that the vaccinated subject may resist or protect itself against an infection by the pathogenic agent against which the vaccination was administered.

Operably linked: Operably linked means that the components described are in a relationship permitting them to function in their intended manner. Thus, for example, a promoter “operably linked” to a nucleic acid means that the promoter and the nucleic acid of a cistron, or more than one cistron, are joined in such a manner that a single cistronic, a single bicistronic, or a single multicistronic messenger RNA (mRNA) may be produced. Protein expression of the messenger RNA may be regulated according to transcriptional/translational elements of the promotor and/or nucleic acid. In another example, an Internal Ribosome Entry Site (IRES) sequence, which is inserted into an expression cassette in an orientation which is upstream (5′) to a cistron means that the IRES sequence and the nucleic acids of the cistron are joined in such a manner that downstream of the IRES, translation of the cistronic mRNA is regulated under the control of the IRES.

Virus-like particle: A virus-like particle is an assembly of viral structural polypeptides, i.e. capsid proteins, which polypeptide assemblies are similar to the authentic virus from which they derive structurally, however, the VLPs do not comprise a virus genome. Enterovirus VLPs do not comprise an RNA genome.

Enterovirus P1: An Enterovirus P1 polypeptide is the primary structural polypeptide of an Enterovirus from which individual structural polypeptides VP0, VP1, VP2, VP3 and VP4 may be cleaved. The order in which the structural polypeptides are arranged on the P1 polypeptide, starting from the N-terminus, is VP0, VP3 and VP1. During encapsidation of the RNA genome in the native virus, VP0 is cleaved into polypeptides VP4 and VP2.

### EXAMPLES

### Example 1. Construction of Chimeric Enterovirus P1 Polypeptides

All members of the genus Enterovirus, including EV-A71, Poliovirus and CV-A16 have a single-stranded, positive sense RNA genome which has a single open reading frame encoding a P1 polypeptide, the P1 polypeptide consisting of the structural polypeptides VP0, VP1, VP2, VP3 and VP4, and which genome encodes several non-structural proteins including the viral proteases 3C and 3CD which are responsible for cleaving the P1 polypeptide into the individual structural polypeptides, VP0, VP3 and VP1, wherein VP0 is eventually cleaved into VP4 and VP2.

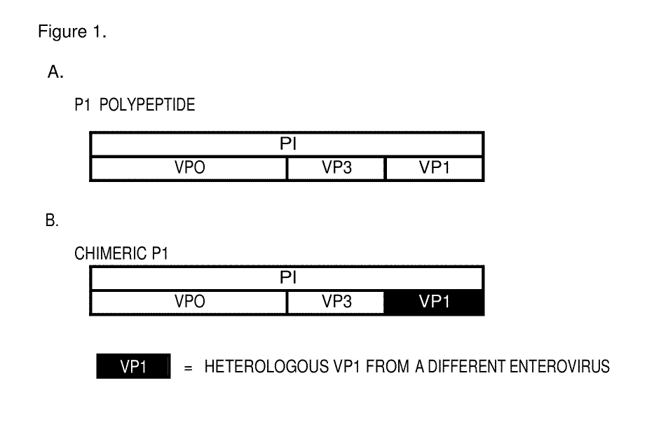

Chimeric P1 polypeptides were constructed, wherein the structural polypeptides VP0 and VP3 comprised in the P1 polypeptide are from one Enterovirus species or serotype, and the VP1 structural polypeptide is derived from a heterologous Enterovirus species or serotype. See FIG. 1A which shows the organization of elements of a P1 polypeptide of Enterovirus. FIG. 1B shows the organization of a chimeric P1 polypeptide having a heterologous VP1 structural polypeptide from an Enterovirus which is different than the Enterovirus from which the VP0 and VP3 structural polypeptides originate.

In an embodiment, chimeric P1 polypeptides were constructed wherein the VP0 and VP3 structural polypeptides originate from Enterovirus EV-A71 and the VP1 structural polypeptide originates from CV-A16.

In another embodiment, chimeric P1 polypeptides were constructed wherein the VP0 and VP3 structural polypeptides originate from Enterovirus CV-A16 and the VP1 structural polypeptide originates from EV-A71.

In a further embodiment, chimeric P1 polypeptides were constructed wherein the VP0 and VP3 structural polypeptides originate from Enterovirus EV-A71 and the VP1 structural polypeptide originates from Poliovirus.

Complete genome sequences of Enterovirus EV-A71 and Enterovirus CV-A16, as well as polioviruses are available in GenBank and are accessible at the National Center for Biotechnology Information (NCBI).

A recombinant DNA molecule encoding such a chimeric P1 polypeptide may be constructed whereby open reading frames which encode Enterovirus structural polypeptides and proteases may be obtained by PCR amplification using suitably designed primers complementary to nucleic acid sequences of Enterovirus. Suitable primers may be designed according to standard techniques from publicly available nucleic acid sequences of Enterovirus such as those complete genome sequences which are available in GenBank and which are accessible at the National Center for Biotechnology Information (NCBI). Moreover, genetic sequences may be synthesized de novo by gene synthesis techniques known in the art.

For example, GenBank accession numbers for the complete genome of EV-A71 include DQ341362, AB204852, AF302996 and AY465356; GenBank accession numbers for the complete genome of the human Enterovirus CV-A16 include KF924762.1; GenBank accession numbers for the complete genome of the human Enterovirus C poliovirus type I genome include V01149 and V01150.

### Example 2. Construction of Expression Cassettes to Obtain Chimeric VLPs

The entry clone pSN01 has been used to generate a recombinant baculovirus harboring an expression cassette for the production of Enterovirus VLPs. The entry clone pSN01 originates from the work described in PCT International Application No. PCT/IB2012/003114, see Example 1, FIG. 1, and is the source of the recombinant baculovirus expression construct, SN07, described in PCT/IB201 2/003114.

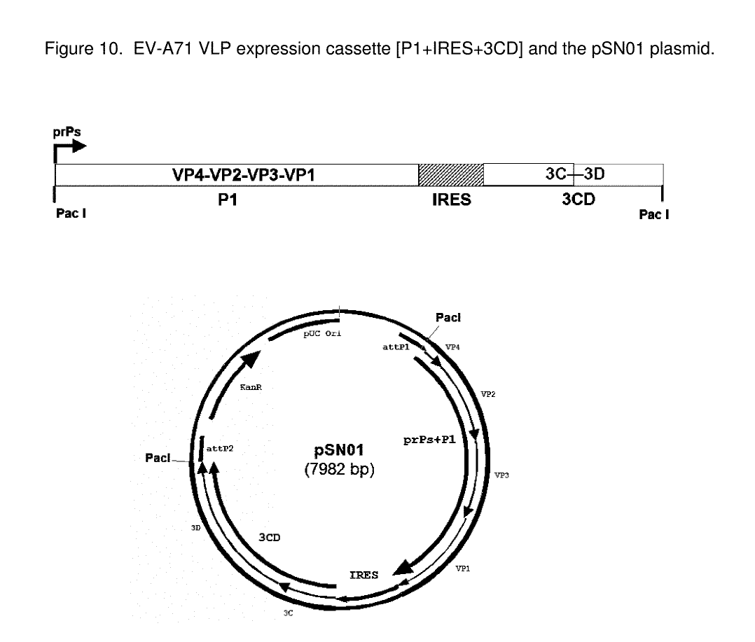

pSN01, depicted in FIG. 10, harbors an expression cassette comprising a nucleic acid encoding an Enterovirus EV-A71 P1 polypeptide, an IRES, and a 3CD protease which derives from EV-A71.

pSN01 may be used to generate a further expression cassette comprising a P1 polypeptide from a different Enterovirus. An example of such an expression cassette may be an expression cassette comprising a P1 polypeptide from Enterovirus CV-A16, an IRES, and a 3CD protease which derives from Enterovirus EV-A71.

Extensive bioinformatics analysis was done to identify a consensus amino acid sequence for Enterovirus CV-A16 P1 and the CV-A16 P1 encoding DNA sequence was codon optimized for species-specific expression.

pSN01 was used to generate an expression cassette where the P1 coding sequence of Enterovirus CV-A16 replaced the coding sequence of EV-A71 in pSN01. The CV-A16 P1 coding sequence was codon optimized for expression in insect cells and the P1 coding sequence was synthesized by methods known to those skilled in the art. The Enterovirus EV-A71 P1 in pSN01 was replaced with the P1 coding sequence of CV-A16 by means known in the art. This construction gave rise to plasmid pSXT6 harboring an expression cassette comprising a CV-A16 P1 polypeptide, an IRES, and a 3CD protease which derives from EV-A71.

For example, the codon optimized CV-A16 P1 gene was synthesized with a BgIII site upstream (5′) of the coding region, a partial IRES and BgII site downstream of the P1 stop codon. The synthesized DNA molecule was cloned into pUC57. The pUC57-CV-A16 P1-IRES(partial)-BgII plasmid was digested with BgII and BgIII and the BgII/BgII DNA fragment containing CV-A16 P1-IRES(partial) was purified. pSN01 was digested with BgII and BgIII and the BgII/BgIII and the vector fragment of pSN01, wherein the EV-A71 P1-IRES(partial) has been removed, was purified and used as the vector for the CV-A16 P1 DNA fragment. The purified CV-A16 P1-IRES(partial) DNA fragment was cloned into the BgII/BgIII digested pSN01 vector giving rise to a CV-A16 P1-IRES-EV-A71 3CD expression construct.

This construction gave rise to plasmid pSXT6 harboring an expression cassette comprising a CV-A16 P1 polypeptide, an IRES, and a 3CD protease which derives from EV-A71.

pSN01 and pSXT6 were used to generate baculovirus expression constructs harboring an expression cassette comprising a chimeric P1 polypeptide, an IRES, and a 3CD protease which derives from EV-A71. To generate recombinant baculoviruses, pSN01 and pSXT6 were digested using the restriction endonuclease NdeI.

The DNA fragments, including the NdeI plasmid vector fragments which resulted from digestion with restriction endonuclease NdeI were purified.

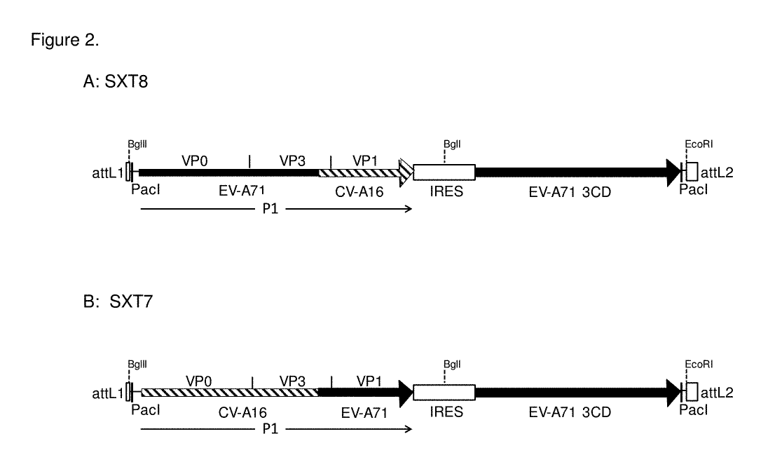

The NdeI fragment which comprises the EV-A71 VP1 coding sequence was ligated into the NdeI digested pSXT6 plasmid vector. In brief, the EV-A71 VP1 coding sequence was swapped for the VP1 coding sequence of CV-A16 in pSXT6. This swap enabled the generation of a bacmid, bacSXT7, comprising an expression cassette comprising a chimeric P1 coding sequence, in particular, the coding sequence for CV-A16 VP0 and VP3, and the coding sequence for VP1 from EV-A71. See FIG. 2B for a graphic representation of the expression cassette of the baculovirus expression construct SXT7.

The NdeI fragment which comprises the VP1 coding sequence from CV-A16 was ligated into the NdeI digested pSN01 plasmid vector. In brief, the CV-A16 VP1 coding sequence was swapped for the VP1 coding sequence of EV-A71 in pSN01. This swap enabled the generation of a bacmid, bacSXT8, comprising an expression cassette comprising a chimeric P1 coding sequence, in particular, the coding sequence for EV-A71 VP0 and VP3, and the coding sequence for VP1 from CV-A16. See FIG. 2A for a graphic representation of the expression cassette of the baculovirus expression construct SXT8.

Rescue of recombinant baculovirus from bacSXT7 or bacSXT8, containing their respective expression cassettes, was carried out according to standard protocols, for example that protocol described in Invitrogen's Guide to Baculovirus Expression Vector Systems (BEVS) and Insect Cell Culture Techniques (Waltham, Mass.). In particular, recombinant bacmid bacSXT7 and recombinant bacmid bacSXT8 were purified using PureLink® HiPure Plasmid Miniprep (ThermoFisher Scientific, Waltham, Mass., USA), and then transfected into Sf9 cells following standard protocols. After 3 days, the supernatant was collected and designated passage 1 (p1) baculovirus stock. This is a small scale low titered baculovirus stock which was amplified by infecting Sf9 cells to generate a passage 2 (p2) baculovirus stock. The passage 2 baculovirus was used to infect Sf9 cells to generate passage 3 (p3) baculovirus stock, which was then used to evaluate expression of chimeric VLPs.

The recombinant baculoviruses which are produced by bacSXT7 are designated as SXT7. The recombinant baculoviruses which are produced by bacSXT8 are designated as SXT8.

A schematic representation of the elements of the expression cassettes for constructs to provide chimeric VLPs is shown in FIGS. 2A and 2B. The nucleotide sequence of the expression cassette of the baculovirus SXT8 construct is provided as SEQ ID NO: 1. The nucleotide sequence of the expression cassette of the baculovirus SXT7 construct is provided as SEQ ID NO: 2.

### Example 3. Expression of Chimeric VLPs in Sf9 Cells Infected with Baculovirus SXT7 Construct or Baculovirus SXT8 Construct

Sf9 cells were infected with baculovirus SXT7 or baculovirus SXT8 at a multiplicity of infection (MOI) of 0.01, and harvested on day 3 post-inoculation. The culture was subjected to centrifugation at 3100×g for 30 min at 15° C. The pellet was washed once with PBS, resuspended in a hypotonic buffer (1.5 mM MgCl₂, 50 mM KCl, 20 mM HEPES) containing 0.1% TritonX100 and an EDTA-free protease inhibitor cocktail (Sigma) and then rocked for 30 min at room temperature (RT). The cell lysate was then clarified by centrifugation at 6600×g for 20 min at 4° C.

As a control, Sf9 cells were infected with baculovirus SN07 (described in PCT International Application No. PCT/IB2012/003114). Baculovirus SN07 originates from the expression clone pSN01 and comprises a native P1 polypeptide from human Enterovirus EV-A71.

The lysates of cells infected with baculovirus SXT7, baculovirus SXT8 or baculovirus SN07 were separated by SDS-PAGE on a 12% gel and then electro-transferred to nitrocellulose membranes. The membranes were blocked in PBS containing 5% skim milk for 1 hr at RT, and then probed with a hyperimmune rabbit polyclonal antibody against EV-A71 VP1 diluted 1:10,000 and incubated overnight at room temperature. Bound antibodies were detected after incubation with anti-rabbit IgG conjugated with horseradish peroxidase (HRP) for 1 hr, followed by 10 min incubation in TMB substrate at room temperature for color development.

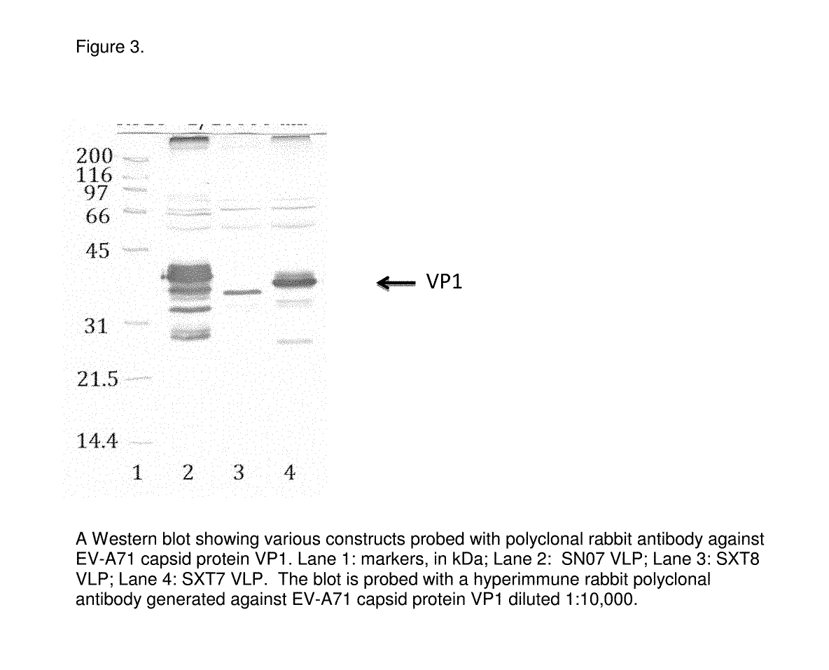

The Western blot in FIG. 3 shows that the EV-A71 VP1 polypeptide of the VLPs obtained from baculovirus SN07 (lane 2), and the EV-A71 VP1 polypeptide of the chimeric VLPs obtained from expression of baculovirus SXT7 (lane 4) are well recognized by the polyclonal rabbit anti-EV-A71 VP1 antibody. The Western blot of chimeric VLPs obtained from expression of baculovirus SXT8 comprising the CV-A16 VP1 (lane 3) shows only a lower molecular weight band which is not as well recognized by the anti-EV-A71 VP1 polyclonal antibody and is characteristic of the cross-reactivity the antibody exhibits against the CV-A16 VP1 polypeptide.

FIG. 3 further demonstrates that the chimeric VLPs which are produced from the expression cassettes exhibit VP1 structural polypeptides, indicating that the chimeric P1 is processed by the EV-A71 3CD protease and that the structural polypeptides are assembled into immunogenic VLPs.

### Example 4. Expression of Chimeric VLPs in Sf9 Cells Infected with Baculovirus SXT7 or Baculovirus SXT8

The lysates of cells infected with baculovirus SXT7, baculovirus SXT8, or baculovirus SN07 were separated by SDS-PAGE on a 12% gel and then electro-transferred to nitrocellulose membranes. The membranes were blocked in PBS containing 5% skim milk for 1 hr at RT, and then probed with a mouse monoclonal antibody against CV-A16 VP1 (F6/2/A1-1/2/A3) overnight at room temperature. Bound antibodies were detected after incubation with anti-mouse IgG conjugated with horseradish peroxidase for 1 hr, followed by 10 min incubation in TMB substrate at room temperature for color development.

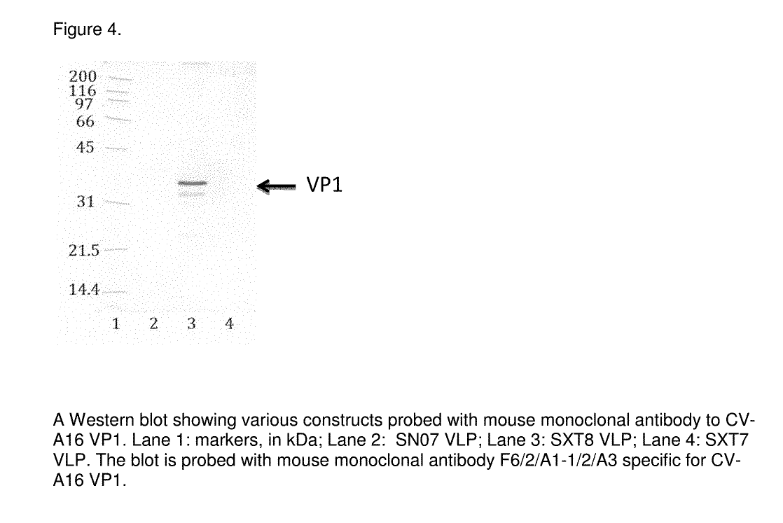

FIG. 4 shows that the EV-A71 VP1 polypeptide of the VLPs obtained from baculovirus SN07 (lane 2) and the EV-A71 VP1 polypeptide of the chimeric VLPs obtained from expression of baculovirus SXT7 (lane 4) are not recognized by the anti-CV-A16 VP1 specific monoclonal antibody F6/2/A1-1/2/A3. However, the CV-A16 VP1 polypeptide of the chimeric VLPs obtained from expression of baculovirus SXT8 (lane 3) clearly shows the presence of CV-A16 VP1 polypeptides (arrow). This demonstrates that the VP1 structural polypeptides of the VLPs obtained from expression of baculovirus SXT8 are CV-A16 VP1 polypeptides.

### Example 5. VLPs Produced by Recombinant Baculovirus SXT7 Generate Antibodies Directed Against EV-A71 Structural Polypeptide VP1 but not Directed Against EV-A71 Structural Polypeptide VP0

Sera from mice immunized with chimeric VLPs obtained by expression of baculovirus SXT7 (containing EV-A71 VP1 structural polypeptide) were tested in an indirect ELISA where wells were coated with an EV-A71 VP1 polypeptide as antigen.

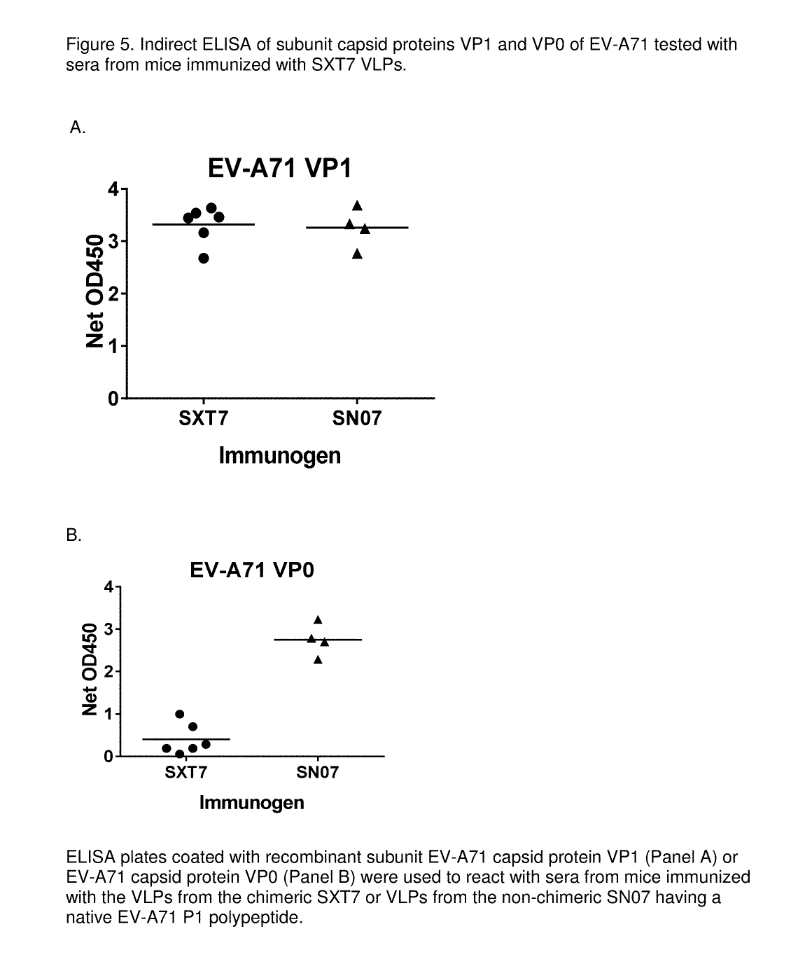

ELISA plates coated with a recombinant subunit EV-A71 VP1 polypeptide antigen (FIG. 5, Panel A) or an EV-A71 VP0 polypeptide antigen (FIG. 5, Panel B) were used to react with sera from mice immunized with the chimeric VLPs obtained from baculovirus SXT7 or sera from mice immunized with VLPs obtained from non-chimeric baculovirus SN07 (described in PCT/IB2012/003114).

FIG. 5 shows that both the sera from mice immunized with VLPs obtained from baculovirus SXT7, as well as the VLPs obtained from baculovirus SN07, comprise antibodies which bind to the EV-A71 VP1 polypeptide antigen, see Panel A. However, only sera from the mice immunized with the non-chimeric SN07 VLPs, and not the chimeric VLPs obtained from baculovirus SXT7, comprise antibodies which bind to EV-A71 VP0 structural polypeptide antigen (Panel B).

Thus, sera from mice immunized with either non-chimeric VLPs obtained from baculovirus SN07 or chimeric VLPs obtained from baculovirus SXT7 comprise antibodies which bind to the EV-A71 VP1 structural polypeptide antigen. The antibodies of the sera from mice immunized with VLPs obtained from baculovirus SXT7 were not able to bind to the EV-A71 VP0 polypeptide antigen for the fact that EV-A71 VP0 polypeptides are not expressed from the SXT7 expression cassette.

### Example 6. Demonstration that VLPs Obtained from Baculovirus SXT8 Exhibit the Functional EV-A71 Neutralizing Epitopes as Found on Native EV-A71 Viruses

Wells were coated with a rabbit polyclonal antibody which is cross-reactive against Enterovirus A VP1 structural polypeptides from EV-A71 and CV-A16. Wells were blocked with bovine serum albumin and lysates from Sf9 cells which were infected with baculovirus SXT8 were diluted 1:10 and were added to the wells and incubated for 1 hr at room temperature. The coating rabbit polyclonal antibody will bind to any VLP containing a VP1 structural polypeptide.

A second antibody was added to the wells to detect epitopes found on the VLPs, such as VP2 structural polypeptides and VP3 structural polypeptides, or any discontinuous epitopes formed from any one or combination of VP2 structural polypeptides and VP3 structural polypeptides.

The detection monoclonal antibodies, E18, E19, and MAB979, were used in 3 separate sets of wells. These monoclonal antibodies were added to the wells and incubated for 1 hr at room temperature. The monoclonal antibodies which bound to the VLPs on the wells were detected by the addition of HRP-conjugated anti-mouse IgG and incubation for 1 hr at room temperature. Color development was achieved by adding TMB substrate for 5 min at room temperature. 0.1N HCl stop solution was added and the absorbance was measured at an optical density of 450 nm (OD₄₅₀).

E18 and E19 are monoclonal antibodies specific for EV-A71 quarternary epitopes and only bind to the virus or VLPs exhibiting these conformational epitopes. MAb979 is a monoclonal antibody which recognizes a linear epitope in the VP2 structural polypeptide of Enterovirus A and should bind to EV-A71 VP2 structural polypeptides, as well as CV-A16 VP2 structural polypeptides.

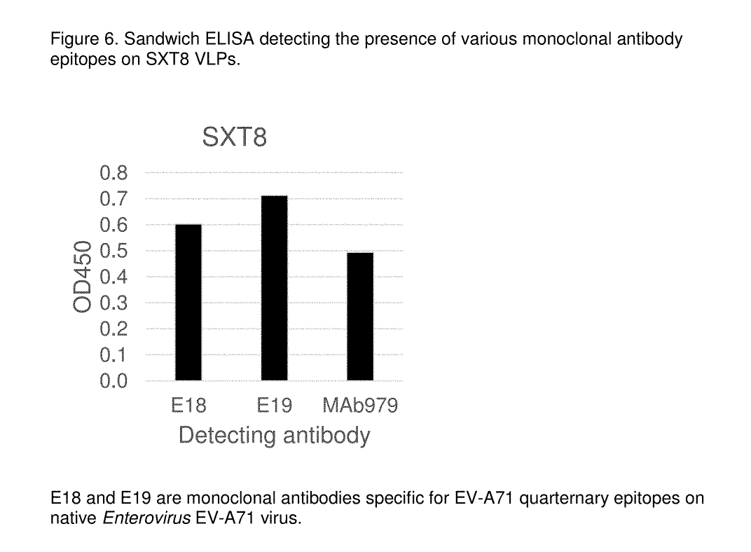

FIG. 6 shows that chimeric VLPs produced by baculovirus SXT8 were bound to the ELISA plate wells via the Enterovirus VP1 polyclonal antibody. Moreover, the figure shows that the chimeric VLPs display the epitopes which are recognized by all 3 monoclonal antibodies, EV18, EV19 and MAb979.

EV18 and EV19 are EV-A71-specific monoclonal antibodies which recognize epitopes formed from the proper assembly of VP0/2 and VP3 of the native EV-A71 virus. The binding footprints of these monoclonal antibodies have been described in the publication PLEVKA, et al.

MAb979 (Merck Millipore) is a commercially available monoclonal antibody which recognizes a linear epitope in the Enterovirus VP2 structural polypeptide. Thus, this example and FIG. 6 demonstrates that the chimeric VLPs generated by the baculovirus SXT8 are assembled particles and that the VP0/2 and VP3 polypeptides of the chimeric VLPs are intact and functional despite the fact that the VP1 structural polypeptides in these VLPs are from CV-A16 and not EV-A71.

It may be concluded that quarternary epitopes of monoclonal antibodies EV18 and EV19 are present on chimeric VLPs obtained from baculovirus SXT8 infected cells, indicating that there is proper assembly of the capsid proteins with intact EV-A71 specific VP0/2 and VP3 neutralizing epitopes.

### Example 7. Demonstration that when VLPs Produced by Recombinant Baculovirus SXT8 are Used to Immunize Mice, No Antibodies are Generated Against EV-A71 VP1

Sera from mice immunized with the chimeric VLPs obtained from baculovirus SXT8 (containing a CV-A16 VP1 structural polypeptide), and sera from mice immunized with the VLPs obtained from baculovirus SN07, were tested in an indirect ELISA as in Example 5, wherein wells of the ELISA plate were coated with a recombinant EV-A71 VP1 structural polypeptide as antigen.

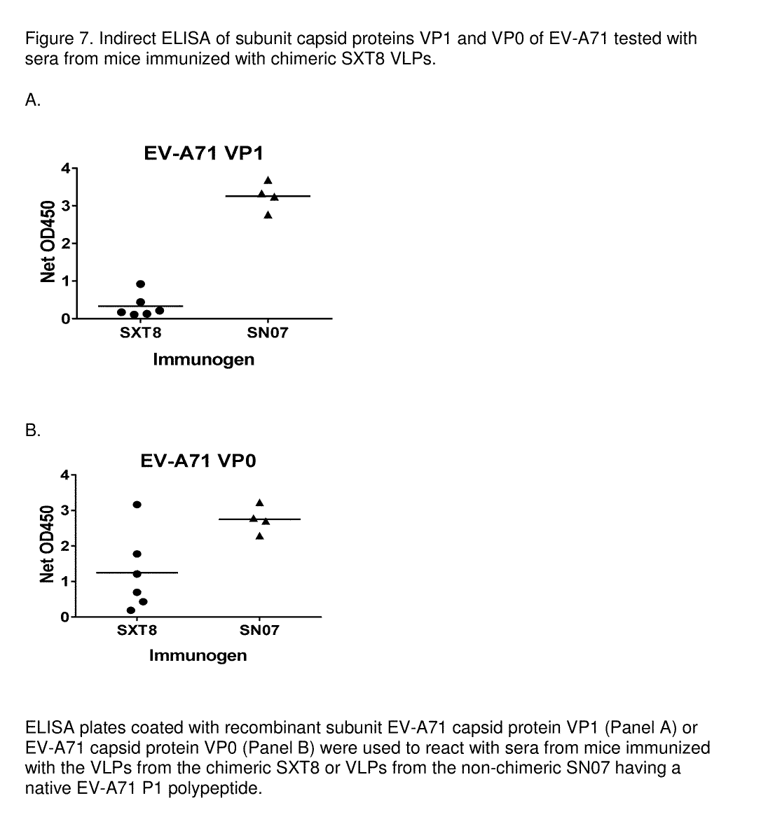

FIG. 7A shows that only sera from mice immunized with non-chimeric EV-A71 VLPs obtained from baculovirus SN07 comprise antibodies which bind to the EV-A71 VP1 polypeptide antigen. The sera from mice immunized with VLPs obtained from SXT8 (which are shown to assemble as VLPs in Example 6 and FIG. 6) were not able to bind to EV-A71 VP1 structural polypeptide antigen for the fact that the VLPs comprise CV-A16 VP1 polypeptides.

Furthermore, it is clear that the same sera from both groups of mice (immunized with VLPs obtained from baculovirus SXT8 or VLPs obtained from baculovirus SN07) comprise antibodies which bind to the EV-A71 VP0 structural polypeptide as shown in FIG. 7B.

### Example 8. Serum Antibodies from Animals Immunized Against Chimeric SXT8 VLPs (Containing a CV-A16 VP1 Structural Polypeptide) Recognize CV-A16 VP1

Wells of ELISA plates were coated with purified VP1 structural polypeptides or VP0 structural polypeptides from both EV-A71 and CV-A16. Serum from animals immunized with chimeric VLPs obtained from cells infected with baculovirus SXT8 (containing a CV-A16 VP1 structural polypeptide) was added to the wells in a dilution series starting at 1:500 to 1:16,000 and was incubated for 1 hour at room temperature. Antibodies which bound to the antigens in the wells were detected by incubating with an HRP-conjugated anti-mouse IgG for 1 hour at room temperature. After washing, color development was accomplished by using a TMB substrate for 5 minutes at room temperature followed by a stopping solution of 0.1N HCl. The absorbance was measured at 450 nm wavelength.

Table 1 shows the titres of antibodies directed at the individual structural polyproteins from CV-A16 and EV-A71 in the serum from animals immunized with SXT8 VLPs.

Not surprisingly, antibodies of the serum recognize VP0 of EV-A71, and not the VP0 structural polypeptide of CV-A16.

Furthermore, the antibodies of the serum recognize VP1 of CV-A16, and do not recognize the VP1 structural polypeptide of EV-A71.

The results shown in Table 1 demonstrate that the chimeric SXT8 VLPs which consist of EV-A71 VP0/2 and VP3 structural polypeptides and VP1 structural polypeptides from CV-A16, do elicit immune responses against the relevant structural polypeptides. Specifically, the serum contains antibodies strongly reactive against the VP0 polypeptides of EV-A71 and not the VP0 polypeptides of CV-A16 since CV-A16 VP0 is not part of the composition of the chimeric VLP. However, the serum also contains antibodies that are strongly reactive against the VP1 polyprotein of CV-A16 but is only very weakly reactive to the VP1 polypeptide of EV-A71.

With regard to VLPs obtained by the baculovirus SXT8, it is demonstrated that the important functional epitopes of EV-A71 are preserved, showing that the VLP is assembled correctly, and the heterologous VP1 polypeptides from CV-A16 are also intact, displayed on the VLP and also elicit strong antibody responses.

### Example 9. The Presence of Antibodies in the Sera of Mice Immunized with Chimeric VLPs

The presence of antibodies against EV-A71 and CV-A16 in sera from animals immunized with chimeric VLPs from baculovirus SXT7 or chimeric VLPs from baculovirus SXT8. Wells were coated with lysates from mock-infected, EV-A71-infected, or CV-A16-infected rhabdomyosarcoma (RD) cells. Sera from animals immunized with chimeric VLPs from baculovirus SXT7 or chimeric VLPs from baculovirus SXT8 were added to the wells at 1/100 dilution. Mice were immunized with the control antigen, FGUS. Wells were washed, and then incubated with HRP-conjugated anti-mouse. TMB substrate was added and Optical Density (OD) was measured at 450 nm. The net OD was calculated by subtracting the OD values of wells coated with lysates from virus-infected RD cells with that of mock-infected RD cells.

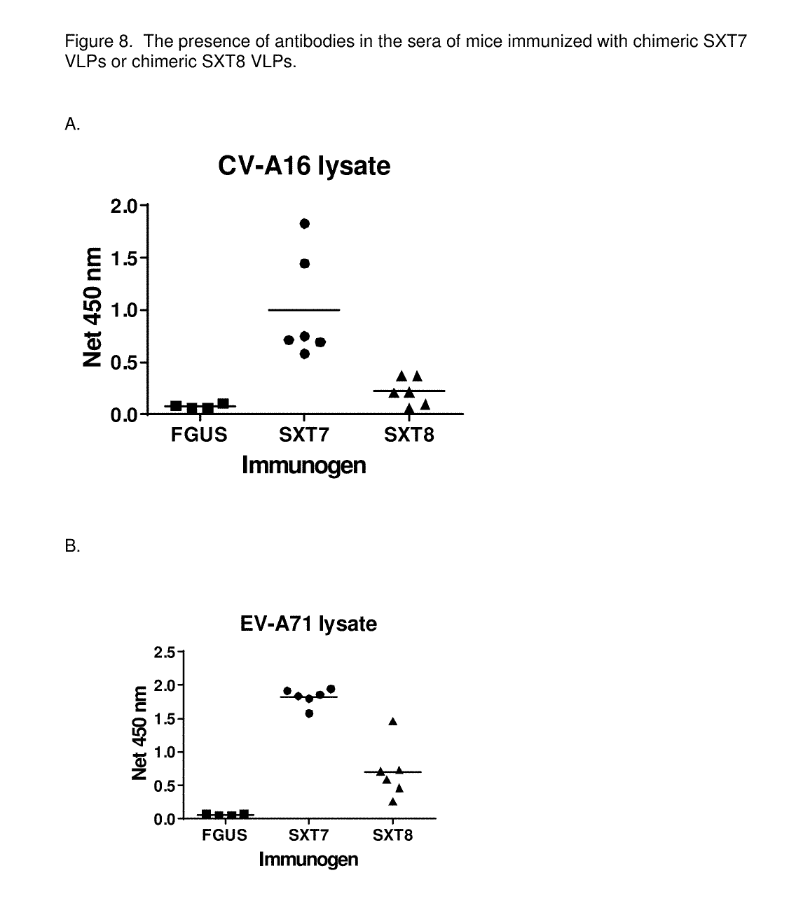

The results in FIG. 8 show that the mice immunized with the control antigen, FGUS, did not bind significantly to the virus-infected cell lysates. FIG. 8 demonstrates that chimeric VLPs obtained by expression of baculovirus SXT7 or baculovirus SXT8 elicit antibody responses directed against both Enterovirus CV-A16 and Enterovirus EV-A71.

Thus, it is demonstrated herein that a bivalent vaccine is achieved with a single immunogen utilizing the chimeric VLPs of this invention. Such chimeric VLPs provide epitopes able to elicit bivalent immune responses that would be protective against infection by both Enterovirus EV-A71 and CV-A16.

The chimeric VLPs provided by this invention thus enable the achievement of a bivalent Enterovirus vaccine in a single VLP, thus making it much more convenient, easier and cheaper to produce a bivalent Enterovirus vaccine without having to mix multiple VLPs, subunits, or antigens together to achieve bivalency.

### Example 10. Construction of Chimeric VLP Having a Poliovirus VP1 Structural Polypeptide Replacing an EV-A71 VP1 Structural Polypeptide

pSN01 was used to generate an expression cassette comprising a P1 polypeptide from Poliovirus (PV), an IRES, and a 3CD protease which derives from EV-A71.

Extensive bioinformatic analysis was done to identify a consensus amino acid sequence for Poliovirus serotype 1 (PV1) P1 polypeptide and the PV1 P1 encoding DNA sequence was codon optimized for species-specific expression. According to methods known to those skilled in the art, the codon optimized PV1 P1 gene was synthesized.

pSN01 was cleaved with restriction endonucleases BgIII and BgIII, thus excising the EV-A71 P1 gene from pSN01, the fragment containing the pSN01 vector backbone was purified. A purified PV1 P1 DNA fragment having compatible ends was then cloned into the digested pSN01 vector backbone giving rise to an expression cassette comprising a PV1 P1 structural polypeptide, an IRES, and an EV-A71 3CD protease. The ligation was screened by PCR and all entry clones were verified by restriction digest. One isolate, pSXT11, was selected.

pSXT11 was used to generate a bacmid, bacSXT11, and the recombinant bacmid was sequence verified.

In a further embodiment, chimeric P1 polypeptides were constructed wherein the VP0 and VP3 structural polypeptides originate from Enterovirus EV-A71 and the VP1 structural polypeptide originates from Poliovirus.

pSN01 (described in PCT/IB2012/003/003114) was used to generate an expression cassette where the VP1 coding sequence of PV-1 replaced the coding sequence of EV-A71 in pSN01.

In brief, the EV-A71 VP1 coding sequence was swapped with the VP1 coding sequence of PV-1 in pSN01. This swap enabled the generation of a bacmid, bacSXT18, comprising an expression cassette comprising chimeric P1 coding sequences, in particular, the coding sequences for EV-A71 VP0 and VP3 polypeptide, and the coding sequence for VP1 polypeptides from PV-1.

The recombinant baculoviruses which are produced by bacSXT11 are designated as SXT11. The recombinant baculoviruses which are produced by bacSXT18 are designated as SXT18.

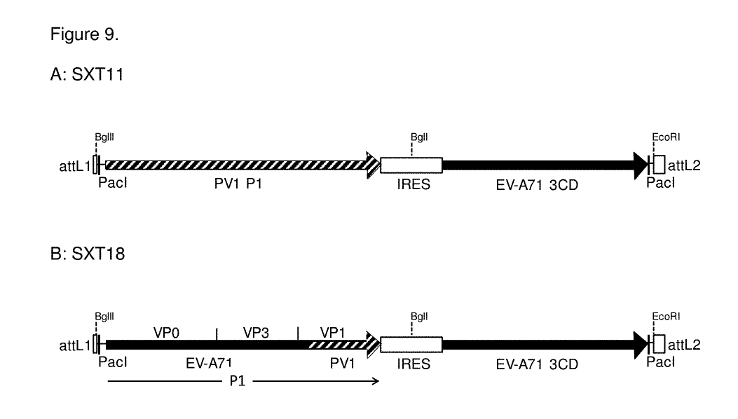

A schematic representation of the elements of the expression cassettes for SXT11 and SXT18 constructs to provide chimeric VLPs is shown in FIGS. 9A and 9B, respectively. The nucleotide sequence of the expression cassette of baculovirus SXT11 is provided as SEQ ID NO: 3. The nucleotide sequence of the expression cassette of baculovirus SXT18 is provided as SEQ ID NO: 4.

### Example 11. Expression of Chimeric VLPs in Sf9 Cells Infected with Baculovirus SXT11 or Baculovirus SXT18

Sf9 cells are infected with baculovirus SXT11 or baculovirus SXT18 at a multiplicity of infection (MOI) of 0.01, and are harvested on day 3 post-inoculation. The culture is subjected to centrifugation at 3100×g for 30 min at 15° C. The pellet is washed once with PBS, resuspended in a hypotonic buffer (1.5 mM MgCl₂, 50 mM KCl, 20 mM HEPES) containing 0.1% TritonX100 and an EDTA-free protease inhibitor cocktail (Sigma) and then rocked for 30 min at room temperature. The cell lysate is then clarified by centrifugation at 6600×g for 20 min at 4° C.

The chimeric VLPs and VP1 structural polypeptides are detected by Western blotting. The lysates of cells infected with baculovirus SXT11 or baculovirus SXT18 are separated by SDS-PAGE on a 12% gel and then electro-transferred to nitrocellulose membranes. The membranes are blocked in PBS containing 5% skim milk for 1 hr at RT, and then probed with a hyperimmune rabbit polyclonal antibody against a VP1 polypeptide and incubated overnight at room temperature. Bound antibodies are detected after incubation with anti-rabbit IgG conjugated with horseradish peroxidase for 1 hr, followed by 10 min incubation in TMB substrate at room temperature for color development.

A band corresponding to Poliovirus VP1 polypeptide is clearly identified, demonstrating that the chimeric VLPs are produced from the expression cassettes and exhibit VP1 structural polypeptides, indicating that the chimeric P1 is processed by the EV-A71 3CD protease and that the structural polypeptides are assembled into VLPs.

### Example 12. VLPs Produced by Recombinant Baculovirus SXT18 Generate Antibodies Directed Against PV1 VP1 Structural Polypeptides

Sera from mice immunized with chimeric VLPs obtained by expression of baculovirus SXT18 (containing Poliovirus-1 VP1 structural polypeptide) are tested in an indirect ELISA where wells are coated with poliovirus VP1 polypeptide as antigen.

ELISA plates coated with a recombinant subunit Poliovirus VP1 polypeptide antigen are used to react with sera from mice immunized with the chimeric VLPs obtained from baculovirus SXT18 or sera from mice immunized with VLPs obtained from non-chimeric baculovirus SXT11.

The sera from mice immunized with VLPs obtained from baculovirus SXT18, as well as the VLPs obtained from baculovirus SXT11, comprise antibodies which bind to the Poliovirus VP1 polypeptide antigen.

### Example 13. The Presence of Antibodies in the Sera of Mice Immunized with Chimeric VLPs

The presence of antibodies against Poliovirus and EV-A71 in sera from animals immunized with chimeric VLPs from baculovirus SXT18 is evaluated utilizing ELISA plates wherein wells are coated with lysates from mock-infected, EV-A71-infected or Poliovirus-infected rhabdomyosarcoma (RD) cells. Sera from animals immunized with chimeric VLPs from baculovirus SXT18 are added to the wells. Mice are immunized with the control antigen. Wells are washed, and then incubated with HRP-conjugated anti-mouse antibody. TMB substrate is added and Optical Density (OD) is measured at 450 nm. The net OD is calculated by subtracting the OD values of wells coated with lysates from virus-infected RD cells with that of mock-infected RD cells.

The results show that the mice immunized with the control antigen did not bind significantly to the virus-infected cell lysates and that chimeric VLPs obtained by expression of baculovirus SXT18 elicit antibody responses directed against both Enterovirus PV-1 and Enterovirus EV-A71.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

### REFERENCES

1. CHUNG, et al., World J Gastroenterol 12(6): 921-927, 2006.
2. CHUNG, et al. Vaccine 26:1855-1862, 2008.
3. CHUNG, et al. Vaccine 28:6951-6957, 2010.
4. ELLIS, Vaccines, Chapter 29, PLOTKIN, et al. (eds) WB Saunders, Philadelphia, at page 571, 1998).
5. Gong M. Zhu H. Zhou J. Yang C. Feng J. Huang X. Ji G. Xu H. Zhu P. Cryo-electron microscopy study of insect cell-expressed enterovirus 71 and coxsackievirus al 6 virus-like particles provides a structural basis for vaccine development. J Virol. 2014 June; 88(11):6444-52. doi: 10.1128/JVI.00200-14. Epub 2014 Mar. 26. PubMed PMID: 24672036; PubMed Central PMCID: PMC4093858.
6. Ku Z. Liu Q., Ye X. Cai Y. Wana X. Shi J. Li D. Jin X. An W. Huana Z. A virus-like particle based bivalent vaccine confers dual protection against enterovirus 71 and coxsackievirus A16 infections in mice. Vaccine. 2014 Jul. 23; 32(34):4296-303. doi: 10.1016/j.vaccine.2014.06.025. Epub 2014 Jun. 17. PubMed PMID: 24950363.
7. LI, et al. New England Journal of Medicine 370(9):829-837, 2014.
8. Li Y., Zhu R. Qian Y., Denq J. The Characteristics of Blood Glucose and WBC Counts in Peripheral Blood of Cases of Hand Foot and Mouth Disease in China: A Systematic Review. PLoS ONE 7(1): e29003; published Jan. 3, 2012.
9. Liu Q. Yan K. Fena Y. Huana X. Ku Z. Cai Y. Liu F. Shi J. Huana Z. A virus-like particle vaccine for coxsackievirus A16 potently elicits neutralizing antibodies that protect mice against lethal challenge. Vaccine. 30 (2012) 6642-6648.
10. MELOEN, et al., J. Gen. Virol. 45:761-763, 1979.
11. PLEVKA, et al. Proc. Natl. Acad. Sci. USA. 111(6):2134-9, 2014.
12. ROSSMANN, et al., Trends in Microbiology, Vol. 10(7) 324-331, 2002.
13. VANSELOW (1987) S. Vet. Bull. 57 881-896.
14. WU, et al., Vaccine 20, 895-904, 2002.
15. ZHU, et al., The Lancet 381:2024-2032, 2013.
16. ZHU, et al. New England Journal of Medicine 370(9):818-828, 2014.

1-36: (canceled)

37. A vaccine comprising a chimeric virus-like particle (VLP) assembled from Enterovirus EV-A71 VP0, VP2, VP4 polypeptides, Enterovirus CV-A16 VP1 polypeptides, and Enterovirus VP3 polypeptides which originate from Enterovirus EV-A71 and have up to 50 amino acids from the C-terminal portion of a VP3 polypeptide from Enterovirus CV-A16.

38. The vaccine of claim 37, wherein the VP2, VP4 and VP3 polypeptides are encoded by nucleotides 107-1801 of the sequence of SEQ ID NO:1, and the VP1 polypeptides are encoded by nucleotides 1802-2695 of SEQ ID NO:1.

39. The vaccine of claim 37, wherein the chimeric Virus-Like Particles (VLPs) are encoded by nucleotides 107-2695 of the sequence of SEQ ID NO:1.

40. The vaccine of claim 37 comprising one or more vaccine adjuvants.

41. A vaccine comprising a chimeric VLP assembled from Enterovirus CV-A16 VP0, VP2, VP4 polypeptides, Enterovirus EV-A71 VP1 polypeptides, and Enterovirus VP3 polypeptides which originate from Enterovirus CV-A16 and have up to 50 amino acids from the C-terminal portion of a VP3 polypeptide from Enterovirus EV-A71.

42. The vaccine of claim 41, wherein the VP2, VP4 and VP3 polypeptides are encoded by nucleotides 107-1801 of the sequence of SEQ ID NO:2, and the VP1 polypeptides are encoded by nucleotides 1802-2695 of SEQ ID NO:2.

43. The vaccine of claim 41, wherein the chimeric VLPs are encoded by nucleotides 107-2695 of the sequence of SEQ ID NO:2.

44. The vaccine of claim 41 comprising one or more vaccine adjuvants.

45. A method of providing an immune response and/or neutralizing immune response against infection by more than one Enterovirus in a subject, the method comprising administering to the subject the vaccine of claim 37 in an amount effective to provide such immune response and/or neutralizing immune response to the more than one Enterovirus.

46. A method of providing an immune response and/or neutralizing immune response against infection by more than one Enterovirus in a subject, the method comprising administering to the subject the vaccine of claim 41 in an amount effective to provide such immune response and/or neutralizing immune response to the more than one Enterovirus.

47. A nucleic acid encoding an expression cassette comprising a nucleic acid encoding a chimeric Enterovirus P1 polypeptide, wherein the nucleic acid encoding the chimeric Enterovirus P1 polypeptide is operably linked to a nucleic acid encoding an Internal Ribosome Entry Site (IRES) and an Enterovirus 3CD protease, wherein the 3CD protease is under the translational control of the IRES.

48. The nucleic acid of claim 47, wherein the expression cassette comprising a nucleic acid encoding a chimeric Enterovirus P1 polypeptide comprises the sequence of SEQ ID NO:1, and wherein the chimeric Enterovirus P1 polypeptide comprises an Enterovirus EV-A71 VP0 polypeptide, an Enterovirus A VP3 polypeptide, and an Enterovirus CV-A16 VP1 polypeptide.

49. The nucleic acid of claim 47, wherein the expression cassette comprising a nucleic acid encoding a chimeric Enterovirus P1 polypeptide comprises the sequence of SEQ ID NO:2, and wherein the chimeric Enterovirus P1 polypeptide comprises an Enterovirus CV-A16 VP0 polypeptide, an Enterovirus A VP3 polypeptide, and an Enterovirus EV-A71 VP1 polypeptide.

50. The nucleic acid of claim 47, wherein the expression cassette comprising a nucleic acid encoding a chimeric Enterovirus P1 polypeptide comprises the sequence of SEQ ID NO:4, and wherein the chimeric Enterovirus P1 polypeptide comprises Enterovirus EV-A71 structural polypeptides VP0 and VP3, and an Enterovirus VP1 structural polypeptide.

51. A method of making a chimeric Enterovirus VLP comprising the steps of culturing a host cell comprising a nucleic acid encoding the expression cassette of claim 48 for a period of time sufficient to produce the chimeric Enterovirus P1 polypeptide and Enterovirus 3CD protease, and to form VLPs, and recovering the VLPs from the host cell.

52. A method of making a chimeric Enterovirus VLP comprising the steps of culturing a host cell comprising a nucleic acid encoding the expression cassette of claim 49 for a period of time sufficient to produce the chimeric Enterovirus P1 polypeptide and Enterovirus 3CD protease, and to form VLPs, and recovering the VLPs from the host cell.

53. A method of making a chimeric Enterovirus VLP comprising the steps of culturing a host cell comprising a nucleic acid encoding the expression cassette of claim 50 for a period of time sufficient to produce the chimeric Enterovirus P1 polypeptide and Enterovirus 3CD protease, and to form VLPs, and recovering the VLPs from the host cell.

54. A vaccine comprising chimeric Virus-Like Particles produced by the method of claim 51.

55. A vaccine comprising chimeric Virus-Like Particles produced by the method of claim 52.

56. A vaccine comprising chimeric Virus-Like Particles produced by the method of claim 53.
